SEQUENCE LISTING (15 sequences)

SEQ ID NO: 1 - RNA, 2002 nt, synthetic construct
  source 1..2002: mol_type = other RNA
gggagagagctcgagcgaacactagccacttgatcacgctgagcgctcgtacaatgaaacacaggtgtgtcagtgctatgcacgttcgaagagctgtatcagcgttcgtgtgaatgagttcaacggagtgttgactaagccggttgctacatttctgtagcacacatagtcaagatttgcaccagacgatactctccctcagtcctgtttatgcaagtcgtcgtagtcctgacgtacttcctaagctcgtcactgtactgatgattccactgatcaagatgcacgtatcttcagtttcctgaagatcggagtaggcactataatcgacaagtaacgcttacgattccatcacgagtgacttacctgaaccataactgacaagggaccacgcagaggtcatactcacaggacttcaaatcttgagtcgggttcgatcatttctgatcgagacaccagtgtgaggtaatcgtacgtcacttgataggagctctaagtagagttgagagcctgttaactagacacgagtaacgaggttagcctgtacgagatatcgggctatagtgcggacacgattgcaccatttctggtgcaacgaaggtgagcatgtatggacaggtcagtgtgactcaagtcgatagtccaagtaggttatcgactcgcatagctcaatgactgtcatcgccagagtatctaggtgtctacctcacgaatcgcgtcgttacatttctgtaacgctcataccgtgctgatctatgggacacgtcgcttattcttgggtcatgacagttgccacaaacaaggcacgacctcacacctgcgaacttcaagcgttaggctgacgttacatgcttgcgtgcactgattcgtttccgaatcagagacctacgaagccagagttcgttcactatcataagtgcactgatgcatttgtgccaacattgaaggcatcgagataaacagccgtcttaatcaagtgagcacctgagatcagcatgattcgtctatttctagacgaatcaacttccattcaggtgccttgctacttaagacgggattaactctcgatgcaacgtgcattggcacaactcgtgatgtgcactttcacactggaacgaactctggcttcgtaggtctgtttgtcatttctgacaaactgcacgcactgttagtacgtcagccacttaaccgaagttcgtcataagtaggtcgtgcgactacgatggcaacttctacttaccaagaataagcgacgtgtcccataatggaagtcggtatgaggtatgactttcgtcatacacgcgattccacaatgtgacacctaacgtttgaggcgatgacctgatacaagctatgcatggttcaaacctacttggactatcgacttgagatgatagtacctgtccaactaacagcaccttcgatacctcgtttccgaggtattcgtgtcctgtgtcaggcccgatattaatgtgtggctaacccttaggaacgtgtctagttaacaggctctcaacgtcatgacgagctcctagtagcaagcgtacgatacattgtgactggtgtctactggatttctccagtaacccgactccgactacaaagtcctgactcattcacctctgcgtggtcccttgtcagttgagtcgatggtaagtcaatgcatcaggaatcgtggttaagtcttgtcgatctgacacactactccgctgtcctgtttccaggacagacgtgcattagcagttgtggaatcatcagtacagtgacgagtcgttactgtacgtcagcttgtttgcgacttgcagttaatcgactgagggtcaaacgtgtctggtgtgtagtcggactatgtgacgttcatttctgaacgtaccggcttagtcaacactccgttgatgagtatgacacgaacgagtcattggctcttcgcttcaatgtagcactgaacttatgatgtttcatacacattacgctcagcgaactgctatggctagtgttcggatcc

SEQ ID NO: 2 - DNA, 2002 bp, synthetic construct
  source 1..2002: mol_type = other DNA
gggagagagctcgagcgaacactagccacttgatcacgctgagcgctcgtacaatgaaacacaggtgtgtcagtgctatgcacgttcgaagagctgtatcagcgttcgtgtgaatgagttcaacggagtgttgactaagccggttgctacatttctgtagcacacatagtcaagatttgcaccagacgatactctccctcagtcctgtttatgcaagtcgtcgtagtcctgacgtacttcctaagctcgtcactgtactgatgattccactgatcaagatgcacgtatcttcagtttcctgaagatcggagtaggcactataatcgacaagtaacgcttacgattccatcacgagtgacttacctgaaccataactgacaagggaccacgcagaggtcatactcacaggacttcaaatcttgagtcgggttcgatcatttctgatcgagacaccagtgtgaggtaatcgtacgtcacttgataggagctctaagtagagttgagagcctgttaactagacacgagtaacgaggttagcctgtacgagatatcgggctatagtgcggacacgattgcaccatttctggtgcaacgaaggtgagcatgtatggacaggtcagtgtgactcaagtcgatagtccaagtaggttatcgactcgcatagctcaatgactgtcatcgccagagtatctaggtgtctacctcacgaatcgcgtcgttacatttctgtaacgctcataccgtgctgatctatgggacacgtcgcttattcttgggtcatgacagttgccacaaacaaggcacgacctcacacctgcgaacttcaagcgttaggctgacgttacatgcttgcgtgcactgattcgtttccgaatcagagacctacgaagccagagttcgttcactatcataagtgcactgatgcatttgtgccaacattgaaggcatcgagataaacagccgtcttaatcaagtgagcacctgagatcagcatgattcgtctatttctagacgaatcaacttccattcaggtgccttgctacttaagacgggattaactctcgatgcaacgtgcattggcacaactcgtgatgtgcactttcacactggaacgaactctggcttcgtaggtctgtttgtcatttctgacaaactgcacgcactgttagtacgtcagccacttaaccgaagttcgtcataagtaggtcgtgcgactacgatggcaacttctacttaccaagaataagcgacgtgtcccataatggaagtcggtatgaggtatgactttcgtcatacacgcgattccacaatgtgacacctaacgtttgaggcgatgacctgatacaagctatgcatggttcaaacctacttggactatcgacttgagatgatagtacctgtccaactaacagcaccttcgatacctcgtttccgaggtattcgtgtcctgtgtcaggcccgatattaatgtgtggctaacccttaggaacgtgtctagttaacaggctctcaacgtcatgacgagctcctagtagcaagcgtacgatacattgtgactggtgtctactggatttctccagtaacccgactccgactacaaagtcctgactcattcacctctgcgtggtcccttgtcagttgagtcgatggtaagtcaatgcatcaggaatcgtggttaagtcttgtcgatctgacacactactccgctgtcctgtttccaggacagacgtgcattagcagttgtggaatcatcagtacagtgacgagtcgttactgtacgtcagcttgtttgcgacttgcagttaatcgactgagggtcaaacgtgtctggtgtgtagtcggactatgtgacgttcatttctgaacgtaccggcttagtcaacactccgttgatgagtatgacacgaacgagtcattggctcttcgcttcaatgtagcactgaacttatgatgtttcatacacattacgctcagcgaactgctatggctagtgttcggatcc

SEQ ID NO: 3 - RNA, 2035 nt, synthetic construct
  source 1..2035: mol_type = other RNA
gggagaggatccgaacactagccatagcagttcgctgagcgtaatgtgtatgaaacatcataagttcagtgctacattgaagcgaagagccaatgactcgttcgtgtcatactcatcaacggagtgttgactaagccggtacgttcagggagggtgaacgtcacatagtccgactacacaccagacacgtttgaccctcagtcgattaactgcaagtcgcaaacaagctgacgtacagtaacgactcgtcactgtactgatgattccacaactgctaatgcacgtctgtcctggggagggcaggacagcggagtagtgtgtcagatcgacaagacttaaccacgattcctgatgcattgacttaccatcgactcaactgacaagggaccacgcagaggtgaatgagtcaggactttgtagtcggagtcgggttactggagggagggtccagtagacaccagtcacaatgtatcgtacgcttgctactaggagctcgtcatgacgttgagagcctgttaactagacacgttcctaagggttagccacacattaatatcgggcctgacacaggacacgaatacctcggggagggcgaggtatcgaaggtgctgttagttggacaggtactatcatctcaagtcgatagtccaagtaggtttgaaccatgcatagcttgtatcaggtcatcgcctcaaacgttaggtgtcacattgtggaatcgcgtgtatgacgggaggggtcatacctcataccgacttccattatgggacacgtcgcttattcttggtaagtagaagttgccatcgtagtcgcacgacctacttatgacgaacttcggttaagtggctgacgtactaacagtgcgtgcagtttgtcagggagggtgacaaacagacctacgaagccagagttcgttccagtgtgaaagtgcacatcacgagttgtgccaatgcacgttgcatcgagagttaatcccgtcttaagtagcaaggcacctgaatggaagttgattcgtctagaaatagacgaatcatgctgatctcaggtgctcacttgattaagacggctgtttatctcgatgccttcaatgttggcacaaatgcatcagtgcacttatgatagtgaacgaactctggcttcgtaggtctctgattcggggagggcgaatcagtgcacgcaagcatgtaacgtcagcctaacgcttgaagttcgcaggtgtgaggtcgtgccttgtttgtggcaactgtcatgacccaagaataagcgacgtgtcccatagatcagcacggtatgagcgttacagggagggtgtaacgacgcgattcgtgaggtagacacctagatactctggcgatgacagtcattgagctatgcgagtcgataacctacttggactatcgacttgagtcacactgacctgtccatacatgctcaccttcgttgcaccagggagggtggtgcaatcgtgtccgcactatagcccgatatctcgtacaggctaacctcgttactcgtgtctagttaacaggctctcaactctacttagagctcctatcaagtgacgtacgattacctcacactggtgtctcgatcagggagggtgatcgaacccgactcaagatttgaagtcctgtgagtatgacctctgcgtggtcccttgtcagttatggttcaggtaagtcactcgtgatggaatcgtaagcgttacttgtcgattatagtgcctactccgatcttcaggggagggctgaagatacgtgcatcttgatcagtggaatcatcagtacagtgacgagcttaggaagtacgtcaggactacgacgacttgcataaacaggactgagggagagtatcgtctggtgcaaatcttgactatgtgtgctacagggagggtgtagcaaccggcttagtcaacactccgttgaactcattcacacgaacgctgatacagctcttcgaacgtgcatagcactgacacacctgtgtttcattgtacgagcgctcagcgtgatcaagtggctagtgttcgctcgag

SEQ ID NO: 4 - RNA, 2038 nt, synthetic construct
  source 1..2038: mol_type = other RNA
gggagagagctcgagcgaacactagccacttgatcacgctgagcgctcgtacaatgaaacacaggtgtgtcagtgctatgcacgttcgaagagctgtatcagcgttcgtgtgaatgagttcaacggagtgttgactaagccggttgctacaccctccctgtagcacacatagtcaagatttgcaccagacgatactctccctcagtcctgtttatgcaagtcgtcgtagtcctgacgtacttcctaagctcgtcactgtactgatgattccactgatcaagatgcacgtatcttcagccctcccctgaagatcggagtaggcactataatcgacaagtaacgcttacgattccatcacgagtgacttacctgaaccataactgacaagggaccacgcagaggtcatactcacaggacttcaaatcttgagtcgggttcgatcaccctccctgatcgagacaccagtgtgaggtaatcgtacgtcacttgataggagctctaagtagagttgagagcctgttaactagacacgagtaacgaggttagcctgtacgagatatcgggctatagtgcggacacgattgcaccaccctccctggtgcaacgaaggtgagcatgtatggacaggtcagtgtgactcaagtcgatagtccaagtaggttatcgactcgcatagctcaatgactgtcatcgccagagtatctaggtgtctacctcacgaatcgcgtcgttacaccctccctgtaacgctcataccgtgctgatctatgggacacgtcgcttattcttgggtcatgacagttgccacaaacaaggcacgacctcacacctgcgaacttcaagcgttaggctgacgttacatgcttgcgtgcactgattcgccctccccgaatcagagacctacgaagccagagttcgttcactatcataagtgcactgatgcatttgtgccaacattgaaggcatcgagataaacagccgtcttaatcaagtgagcacctgagatcagcatgattcgtctatttctagacgaatcaacttccattcaggtgccttgctacttaagacgggattaactctcgatgcaacgtgcattggcacaactcgtgatgtgcactttcacactggaacgaactctggcttcgtaggtctgtttgtcaccctccctgacaaactgcacgcactgttagtacgtcagccacttaaccgaagttcgtcataagtaggtcgtgcgactacgatggcaacttctacttaccaagaataagcgacgtgtcccataatggaagtcggtatgaggtatgacccctcccgtcatacacgcgattccacaatgtgacacctaacgtttgaggcgatgacctgatacaagctatgcatggttcaaacctacttggactatcgacttgagatgatagtacctgtccaactaacagcaccttcgatacctcgccctccccgaggtattcgtgtcctgtgtcaggcccgatattaatgtgtggctaacccttaggaacgtgtctagttaacaggctctcaacgtcatgacgagctcctagtagcaagcgtacgatacattgtgactggtgtctactggaccctccctccagtaacccgactccgactacaaagtcctgactcattcacctctgcgtggtcccttgtcagttgagtcgatggtaagtcaatgcatcaggaatcgtggttaagtcttgtcgatctgacacactactccgctgtcctgccctccccaggacagacgtgcattagcagttgtggaatcatcagtacagtgacgagtcgttactgtacgtcagcttgtttgcgacttgcagttaatcgactgagggtcaaacgtgtctggtgtgtagtcggactatgtgacgttcaccctccctgaacgtaccggcttagtcaacactccgttgatgagtatgacacgaacgagtcattggctcttcgcttcaatgtagcactgaacttatgatgtttcatacacattacgctcagcgaactgctatggctagtgttcggatcc

SEQ ID NO: 5 - RNA, 2035 nt, synthetic construct
  source 1..2035: mol_type = other RNA
gggagaggatccgaacactagccatagcagttcgctgagcgtaatgtgtatgaaacatcataagttcagtgctacattgaagcgaagagccaatgactcgttcgtgtcatactcatcaacggagtgttgactaagccggtacgttcaaaagaaatgaacgtcacatagtccgactacacaccagacacgtttgaccctcagtcgattaactgcaagtcgcaaacaagctgacgtacagtaacgactcgtcactgtactgatgattccacaactgctaatgcacgtctgtcctgaaagaaacaggacagcggagtagtgtgtcagatcgacaagacttaaccacgattcctgatgcattgacttaccatcgactcaactgacaagggaccacgcagaggtgaatgagtcaggactttgtagtcggagtcgggttactggaaaagaaatccagtagacaccagtcacaatgtatcgtacgcttgctactaggagctcgtcatgacgttgagagcctgttaactagacacgttcctaagggttagccacacattaatatcgggcctgacacaggacacgaatacctcgaaagaaacgaggtatcgaaggtgctgttagttggacaggtactatcatctcaagtcgatagtccaagtaggtttgaaccatgcatagcttgtatcaggtcatcgcctcaaacgttaggtgtcacattgtggaatcgcgtgtatgacaaagaaagtcatacctcataccgacttccattatgggacacgtcgcttattcttggtaagtagaagttgccatcgtagtcgcacgacctacttatgacgaacttcggttaagtggctgacgtactaacagtgcgtgcagtttgtcaaaagaaatgacaaacagacctacgaagccagagttcgttccagtgtgaaagtgcacatcacgagttgtgccaatgcacgttgcatcgagagttaatcccgtcttaagtagcaaggcacctgaatggaagttgattcgtctagaaatagacgaatcatgctgatctcaggtgctcacttgattaagacggctgtttatctcgatgccttcaatgttggcacaaatgcatcagtgcacttatgatagtgaacgaactctggcttcgtaggtctctgattcgaaagaaacgaatcagtgcacgcaagcatgtaacgtcagcctaacgcttgaagttcgcaggtgtgaggtcgtgccttgtttgtggcaactgtcatgacccaagaataagcgacgtgtcccatagatcagcacggtatgagcgttacaaaagaaatgtaacgacgcgattcgtgaggtagacacctagatactctggcgatgacagtcattgagctatgcgagtcgataacctacttggactatcgacttgagtcacactgacctgtccatacatgctcaccttcgttgcaccaaaagaaatggtgcaatcgtgtccgcactatagcccgatatctcgtacaggctaacctcgttactcgtgtctagttaacaggctctcaactctacttagagctcctatcaagtgacgtacgattacctcacactggtgtctcgatcaaaagaaatgatcgaacccgactcaagatttgaagtcctgtgagtatgacctctgcgtggtcccttgtcagttatggttcaggtaagtcactcgtgatggaatcgtaagcgttacttgtcgattatagtgcctactccgatcttcagaaagaaactgaagatacgtgcatcttgatcagtggaatcatcagtacagtgacgagcttaggaagtacgtcaggactacgacgacttgcataaacaggactgagggagagtatcgtctggtgcaaatcttgactatgtgtgctacaaaagaaatgtagcaaccggcttagtcaacactccgttgaactcattcacacgaacgctgatacagctcttcgaacgtgcatagcactgacacacctgtgtttcattgtacgagcgctcagcgtgatcaagtggctagtgttcgctcgag

SEQ ID NO: 6 - RNA, 2038 nt, synthetic construct
  source 1..2038: mol_type = other RNA
gggagagagctcgagcgaacactagccacttgatcacgctgagcgctcgtacaatgaaacacaggtgtgtcagtgctatgcacgttcgaagagctgtatcagcgttcgtgtgaatgagttcaacggagtgttgactaagccggttgctacatttcttttgtagcacacatagtcaagatttgcaccagacgatactctccctcagtcctgtttatgcaagtcgtcgtagtcctgacgtacttcctaagctcgtcactgtactgatgattccactgatcaagatgcacgtatcttcagtttctttctgaagatcggagtaggcactataatcgacaagtaacgcttacgattccatcacgagtgacttacctgaaccataactgacaagggaccacgcagaggtcatactcacaggacttcaaatcttgagtcgggttcgatcatttcttttgatcgagacaccagtgtgaggtaatcgtacgtcacttgataggagctctaagtagagttgagagcctgttaactagacacgagtaacgaggttagcctgtacgagatatcgggctatagtgcggacacgattgcaccatttcttttggtgcaacgaaggtgagcatgtatggacaggtcagtgtgactcaagtcgatagtccaagtaggttatcgactcgcatagctcaatgactgtcatcgccagagtatctaggtgtctacctcacgaatcgcgtcgttacatttcttttgtaacgctcataccgtgctgatctatgggacacgtcgcttattcttgggtcatgacagttgccacaaacaaggcacgacctcacacctgcgaacttcaagcgttaggctgacgttacatgcttgcgtgcactgattcgtttctttcgaatcagagacctacgaagccagagttcgttcactatcataagtgcactgatgcatttgtgccaacattgaaggcatcgagataaacagccgtcttaatcaagtgagcacctgagatcagcatgattcgtctatttctagacgaatcaacttccattcaggtgccttgctacttaagacgggattaactctcgatgcaacgtgcattggcacaactcgtgatgtgcactttcacactggaacgaactctggcttcgtaggtctgtttgtcatttcttttgacaaactgcacgcactgttagtacgtcagccacttaaccgaagttcgtcataagtaggtcgtgcgactacgatggcaacttctacttaccaagaataagcgacgtgtcccataatggaagtcggtatgaggtatgactttctttgtcatacacgcgattccacaatgtgacacctaacgtttgaggcgatgacctgatacaagctatgcatggttcaaacctacttggactatcgacttgagatgatagtacctgtccaactaacagcaccttcgatacctcgtttctttcgaggtattcgtgtcctgtgtcaggcccgatattaatgtgtggctaacccttaggaacgtgtctagttaacaggctctcaacgtcatgacgagctcctagtagcaagcgtacgatacattgtgactggtgtctactggatttcttttccagtaacccgactccgactacaaagtcctgactcattcacctctgcgtggtcccttgtcagttgagtcgatggtaagtcaatgcatcaggaatcgtggttaagtcttgtcgatctgacacactactccgctgtcctgtttctttcaggacagacgtgcattagcagttgtggaatcatcagtacagtgacgagtcgttactgtacgtcagcttgtttgcgacttgcagttaatcgactgagggtcaaacgtgtctggtgtgtagtcggactatgtgacgttcatttcttttgaacgtaccggcttagtcaacactccgttgatgagtatgacacgaacgagtcattggctcttcgcttcaatgtagcactgaacttatgatgtttcatacacattacgctcagcgaactgctatggctagtgttcggatcc

SEQ ID NO: 7 - RNA, 2310 nt, synthetic construct
  source 1..2310: mol_type = other RNA
gggagagagctcgagcgaacactagccacttgatcactcgtgcttctcgtacatggaagcccaggtgtggagataaggtcttaggttttcctaagatgagcgtttaacgtgcatcttgactctgatacaattccatagactcattcatgtcgcatacctctgttttcagaggtaaccacgggtgtagtcgactgcgtagtcaaacgtcgatctacagctaatcgaggctttctccatcgttttcgatggaatgattcggcaaactaggacacgagagtaacgaaggaatgtacaatcgactcgacattcaaccagcttttgctggttggatcgtcgtgtgtcaggcgttgtccacttaaccagtagaccctaaacagtcactgacgactatgcttttgcatagttatggtacaatgcatcatgtacgaggagtcgatgccatatagtgagtatgcacgtgagagacgaacttttgttcgtcttgacttactacctcactatgctagatcaagtgatcgggatttcataagtgttaagggcatcgctatttttagcgatcgcaagtggtgctgatcacgaccatatagaagtcatggaaactgatcaagtagatggtgtggcttgttttcaagccacttgcgtatgtcatgacaaagccttagtagcaagatgggacttgcacgttctcattggcgcataggttttcctatgcttcaggagttacatgctgtacctcaatggttcatccatgccgcgactacagcagcaacaatgatcgttttcgatcattcatagggtcagtgtgacgtgaatcgtaacgcttatgaaccactagtttgcagggttgtatatgcgttttcgcatatcactccactggcctaattcgataccttcctaagctccgcgacctgacacagttagctctgtcgaacttttgttcgacaaggcacttactatcatgttcgatacagagtatctgactgtgtgatgcataagcaatggccacagcttttgctgtggaccagcagtactaacagcttaagagagtcattgtgttatgtcgtgaggtacagctcacgtcagatcttttgatctgactggtcgaatctacgtactggttcaataatgtgtcgtaatcggatcagcatgattcgtctatttctagacgaatcaacttccatcgattacgtgtacgagttgaaccaggtcatgacttcgaccaaccacctgttttcaggtggtgtgagctgacattgtggacataacatgtatcagctcttaagagcatgtaactgctggtaacgacgttttcgtcgttccattgcttctcgtgatcacagtcaacgtttgagtatcgaactcacactgaagtgcctctggtatgttttcataccaggagctaacgcactatagtcgcggagtcgttactggtatcgaattaggccagtggagtgctttagcttttgctaaagacaaccctgtcgtagtctggttcatggttaagtcgattcacgatgatagtaccctatggatcttacttttgtaagatcgttgctgcacaatcttcggcatggaatcgactctgaggtacctgttagtactcctgaaatgtcacttttgtgacatgccaatgaggttcaatgagtcccattcacttgataaggcttttacgtagaatacgcaactactaacttttgttagtagaccatctatagcagttgtttccatgacttctatatggtcgtatggaagtccacttgcgtgaacgatttttcgttcagcccttaaccacacctgaatcccgacttgctactctagcatacacaatgtgtaagtcaggctctggttttccagagccctcacgtggaatgagtctatatggctgaaccatctcgtacaatcacgagtgtaccatagtatgagttttctcataccgtcagtgagattagctggtctactaagcgttaggacaacgctatagtgccgacgatctctagtcgttttcgactagaaatgtcgagtcgattgtacattcctcttaggaactcgtgtcgactacgaccgaatcatagtaaccttttggttactgaaagcctcctgtttaggtagatcggatactctctacgcagtaagattgtcccgtggtagatcctcttttgaggatcttgcgacatcatactcactatggaatcaatgactagtcaagacattgaacaaacgctcaagtcatcttttgatgactccttatctcacttatgaggcttccaacacattaaagcacgagaactgctatggctagtgttcggatcc

SEQ ID NO: 8 - RNA, 2088 nt, synthetic construct
  source 1..2088: mol_type = other RNA
gggagagagctcgagcgaacactagccacttgatcaactggacatacattgtgttcatgcctgtagtcgatctgaatggcactatatccataaacacacgtcgctgcgtaggactacgacgaaaatgagagtatccacagcatacatactcagatagtggtcataagtctgctgtgcatcgagaacagaagaagtagcaaggagctcatcacagtcgttttcgactgtgcagttgtagattagctaccggttgatgaaaccattaatgaagcccacccggactgatttcctaagaactcttgtgatactctccttggttagtcattgcgggagtgaacacattagtgaacatgaagccataatgcgatgacttccatggagcaaacacaatgtagaaactcaatggttcagccggattctaagtcgttttcgacttaggatagtagggagatggacgattcggacttaacctagcttgtacaatcttatctaggcacattgaactagaagggtaatgtgtggacctttctactttgtcaatatccgtcatgactcagggactgaatgagtgctgatcgcagtgtgaccatccccgggtttcatagcctggggaaaacaagcccttacagtagcacttttgtgctaccgttcggtcctgtttagtttgcagaactaacagtgccaaagtacttatgactcccttgatatagactatgcttaggtcatgacaggagctgctgatacaggaaccaatctcgtgatgagtaacctatagtgcaactacttcatacgcatgtaacaaaggttaagtgccgacttcaagtcgatgtgccgacagtaacacttttgtgttactcatacttgtagcagtttcacagaatctacacatactctggaatggaagtgcgtgtgtttgcacgttgctgattgatcacgagacaagggaactagtttgtcaatttcacaagcacctacgacgtcttaggaagtaagtcgactatcatcccttgttcctgttagtaagcactgtcaagtgatactgctctatttctagagcagtacttgctaccagtgctttacatgctgaacaagggtcacactgcgacttacagtaacgaacgtcgtaggtgcttgtgaaattgatcgtagtcttcccttgtatgcatcacaatcagcgttcaatgaacacacgctgctgatctccagagtatgtgtagattctgtgatgatcaagcaagtatgctgcgtagttttctacgcaggtcggcactgaaccatgaagtcggctaacgctttttgttacatgcgtatgaagtagtttgtgtcagggttactctgatgcatattggttcccaatgactcagctccttctacgtactaagcatagtctatatcaagggagcaggtgtgactttggcaagcatgtatctgcaaaagctaatcgaccgaacgtcggtcgttttcgaccgatgtaagggcttgttttccccaggctccgtatgacggggatggatgatagtcgatcagctgagtatgagtccctgatacgtagaggatattgacaaagtagaaaggtcctgtacgagcccttctaaacgtgcatgcctagatcgactacaacaagctaaagcgttaccgaatcgtccatctccctactatcgtcacaacttttgttgtgacaatccggcatcgactttgagtttcttacctcactttgctccgatcagcacatcgcattatggcttcatgttcacctcgtacatcactcccgtgtatcagaaccaaggtcaaacgtacaagagtttcgttactatcagtccgggtgggcttcattaattcatacggcaaccggtctaaacagtacaactgcgttacccttttgggtaacgatgagctctcacttgattcttctgttctcgatgcacagcagcacacctgccactatcactcattctatgctgtgacgtttgacattttcgcaaactagctacgcagcgacgtgtgtttatggactgacacacattcagataagattgtggcatgaagtgaggtaatgtccagtaactgctatggctagtgttcggatcc

SEQ ID NO: 9 - RNA, 1838 nt, synthetic construct
  source 1..1838: mol_type = other RNA
gggagaggatccgaacactagccatagcagttcgctgagcgtaatgtgtatgaaacatcataagttcagtgctacattgaagcgaagagccaatgactcgttcgtgtcatactcatcaacggagtgttgactaagccgaaaaaacatagtccgactacacaccagacacgtttgaccctcagtcgattaactgcaagtcgcaaacaagctgacgtacagtaacgactcgtcactgtactgatgattccacaactgctaatgcacgaaaaaaggagtagtgtgtcagatcgacaagacttaaccacgattcctgatgcattgacttaccatcgactcaactgacaagggaccacgcagaggtgaatgagtcaggactttgtagtcggagtcggaaaaaacaccagtcacaatgtatcgtacgcttgctactaggagctcgtcatgacgttgagagcctgttaactagacacgttcctaagggttagccacacattaatatcgggcctgacacaggacacgaaaaaagaaggtgctgttagttggacaggtactatcatctcaagtcgatagtccaagtaggtttgaaccatgcatagcttgtatcaggtcatcgcctcaaacgttaggtgtcacattgtggaatcgcaaaaaacataccgacttccattatgggacacgtcgcttattcttggtaagtagaagttgccatcgtagtcgcacgacctacttatgacgaacttcggttaagtggctgacgtactaacagtgcgtgcaaaaaagacctacgaagccagagttcgttccagtgtgaaagtgcacatcacgagttgtgccaatgcacgttgcatcgagagttaatcccgtcttaagtagcaaggcacctgaatggaagttgattcgtctagaaatagacgaatcatgctgatctcaggtgctcacttgattaagacggctgtttatctcgatgccttcaatgttggcacaaatgcatcagtgcacttatgatagtgaacgaactctggcttcgtaggtcaaaaaagcacgcaagcatgtaacgtcagcctaacgcttgaagttcgcaggtgtgaggtcgtgccttgtttgtggcaactgtcatgacccaagaataagcgacgtgtcccatagatcagcacggtatgaaaaaagcgattcgtgaggtagacacctagatactctggcgatgacagtcattgagctatgcgagtcgataacctacttggactatcgacttgagtcacactgacctgtccatacatgctcaccttcaaaaaacgtgtccgcactatagcccgatatctcgtacaggctaacctcgttactcgtgtctagttaacaggctctcaactctacttagagctcctatcaagtgacgtacgattacctcacactggtgaaaaaaccgactcaagatttgaagtcctgtgagtatgacctctgcgtggtcccttgtcagttatggttcaggtaagtcactcgtgatggaatcgtaagcgttacttgtcgattatagtgcctactccaaaaaacgtgcatcttgatcagtggaatcatcagtacagtgacgagcttaggaagtacgtcaggactacgacgacttgcataaacaggactgagggagagtatcgtctggtgcaaatcttgactatgaaaaaacggcttagtcaacactccgttgaactcattcacacgaacgctgatacagctcttcgaacgtgcatagcactgacacacctgtgtttcattgtacgagcgctcagcgtgatcaagtggctagtgttcgctcgagctctctccctttagtgagggttaattaagct

SEQ ID NO: 10 - RNA, 1838 nt, synthetic construct
  source 1..1838: mol_type = other RNA
gggagagagctcgagcgaacactagccacttgatcacgctgagcgctcgtacaatgaaacacaggtgtgtcagtgctatgcacgttcgaagagctgtatcagcgttcgtgtgaatgagttcaacggagtgttgactaagccgttttttcatagtcaagatttgcaccagacgatactctccctcagtcctgtttatgcaagtcgtcgtagtcctgacgtacttcctaagctcgtcactgtactgatgattccactgatcaagatgcacgttttttggagtaggcactataatcgacaagtaacgcttacgattccatcacgagtgacttacctgaaccataactgacaagggaccacgcagaggtcatactcacaggacttcaaatcttgagtcggttttttcaccagtgtgaggtaatcgtacgtcacttgataggagctctaagtagagttgagagcctgttaactagacacgagtaacgaggttagcctgtacgagatatcgggctatagtgcggacacgttttttgaaggtgagcatgtatggacaggtcagtgtgactcaagtcgatagtccaagtaggttatcgactcgcatagctcaatgactgtcatcgccagagtatctaggtgtctacctcacgaatcgcttttttcataccgtgctgatctatgggacacgtcgcttattcttgggtcatgacagttgccacaaacaaggcacgacctcacacctgcgaacttcaagcgttaggctgacgttacatgcttgcgtgcttttttgacctacgaagccagagttcgttcactatcataagtgcactgatgcatttgtgccaacattgaaggcatcgagataaacagccgtcttaatcaagtgagcacctgagatcagcatgattcgtctatttctagacgaatcaacttccattcaggtgccttgctacttaagacgggattaactctcgatgcaacgtgcattggcacaactcgtgatgtgcactttcacactggaacgaactctggcttcgtaggtcttttttgcacgcactgttagtacgtcagccacttaaccgaagttcgtcataagtaggtcgtgcgactacgatggcaacttctacttaccaagaataagcgacgtgtcccataatggaagtcggtatgttttttgcgattccacaatgtgacacctaacgtttgaggcgatgacctgatacaagctatgcatggttcaaacctacttggactatcgacttgagatgatagtacctgtccaactaacagcaccttcttttttcgtgtcctgtgtcaggcccgatattaatgtgtggctaacccttaggaacgtgtctagttaacaggctctcaacgtcatgacgagctcctagtagcaagcgtacgatacattgtgactggtgttttttccgactccgactacaaagtcctgactcattcacctctgcgtggtcccttgtcagttgagtcgatggtaagtcaatgcatcaggaatcgtggttaagtcttgtcgatctgacacactactccttttttcgtgcattagcagttgtggaatcatcagtacagtgacgagtcgttactgtacgtcagcttgtttgcgacttgcagttaatcgactgagggtcaaacgtgtctggtgtgtagtcggactatgttttttcggcttagtcaacactccgttgatgagtatgacacgaacgagtcattggctcttcgcttcaatgtagcactgaacttatgatgtttcatacacattacgctcagcgaactgctatggctagtgttcggatcctctccctatagtgagtcgtattagaatt

SEQ ID NO: 11 - RNA, 2632 nt, synthetic construct
  source 1..2632: mol_type = other RNA
gggagagagctcgagcgaacactagacttgatcacttcgtttagcgaaatcgactctggatagtacattgaacgtgactcctcataagtgctttgaagtaatgtgtaggctatagatcagcacggtcacttaacattaggcaacgctactcaatgttttcattgagtgctacactgtcatgactgtgcatgacttgctacagtttgtcctgatacatacagatcccgactacagtgcgacagattagcttgccttcatgtttgccgttttcggcaaaccacacgcattgcagatgcgccacgacttaggaagaatgcatgacttaaccacatcagatgatgcatcccgatagacatactcaagactagttacctcactagcaacctggtgcgatgttcaaagctacgtcgttttcgacgtagcatggcgctacatgcttaaagaataacgtttgaaggcggcatatagtgcatatggccgatgaaaccggtggctaagttgactttttcgagagaacagggttttccctgttcgtagtggtacactcaggtataaaagagtgctatctctaatctgataactggccactggtggtatctcggtttgatgactacgacattgttcactatcataatgctagcctgttcacaccgacagtctcaatgttttcattgagagtacgagtgaacgtccacttatctgatgatagtttgatctcactaacagcgatagcctgtgaggtacaatatcctacgtagatcctcttggtgctgatcccaaagtcttatcgagatctcatagttaaccagttttctggttaagagagcgacctcgtacaacctatacgtagcaaggcgactgacgaatgagtcgtggttatcaaacgtaagttaggcctagtttggacattcatacatgagttttctcatgtagggcagtgagttgatatgtccacctagataccaaatcctctctgacacagtttcatgtatgcatcaacccttcgagtcattggttatcaccacttatgatatatcccagtcagtcgttcgatcgtctgcgtgttttcacgcagagaattgcgctgcacgttcatgtatttgtagtcggaagatagctaacgcttcacgtgggggtttcatatagtgtcgtgtagactcaggatcgacgtgatgttttcatcacgtgtaggtaagtcaccatattttggaaatagcactgtgtgttgtacaggagagtccgtaattcctaagcacgtcttctgtttaggtttggagcgagtcgatacctgcgaccgctatgatcaaggtctccatctatttctagatggagactagcagtttagcggtcgcaggttgaaccatgctccaaacagctaatcaagacgtgtcgttactttacggactagtcaactcaacacactcctgatgttccaaaatatggtgacttacctaccctatatcttttgatataggcgatcctgcagttatccgacactatccgtatgacccacgtgggttaagtgctatcttcaagattgtaatacatggttcaatggcgcaattcgacattacttttgtaatgtccgatcgaacgactgactgggatatacaggtgtgggtgataactgtatcagcgaagggtatcacgagacatgaaacgcactataagaggattgacacctaaggtggacatatcaactcactgcccgatgcatcttttgatgcatctgaatgtctcgtagtcgcctaacttgatactcttaaccacgtgagtatggtcagtcgctcacttgagtataggtacacattagtcgctctccaaagcacttttgtgctttgctatgagatctcgataagactttggatggaagtccaagaggagtcatgacggatattgacattgtgaggctatcgagcatgtagagatcaacagtgtgacagataagtggacgttcactcgtacacaccaccttttggtggtgtctgtcggtgtgaacaggctagcatttcacactggaacaatgcaaactagatcaaaccgtaggtgtcccagtggcagtctacaagattagagcatcaggacttttatacctgagtgtaccactacttgctagcttttgctagcaatctcgaaaactcctgtatagccacctcatacggcggccatattgtgtcagtgccgcctagagtatctattctttactgttagtgcgccatggtgacttcttttgaagtcacctttgaacatcgcaccaggttgctacacaatgtactagtctactcattctctatcgggctcgtgattctgatgtaagcgttacatgcattcagtaacgatcgtggcgcatctgcaatgcgtgtggagtcatcttttgatgactcatgaaggcactaaacagtgtcgcacacaatcttggatctgtacaatgactgacaaacttcaagtgatcatgcacatctacgtaagtgtagctgcgcaccttttggtgcgcaagcgttgcctaatgttaagtgaccgacttccattatagccttgtacgagcttcaaagccacacctgggagtcacaacgtgcatactatccaatggttcattcgctaaacgaagaactgctatctagtgttcggatcc

SEQ ID NO: 12 - RNA, 1684 nt, synthetic construct
  source 1..1684: mol_type = other RNA
gggagattactcataagggctggcttggttcactaggagctagttgggtagcccgtcaccagtgcgtacagcccgttcatccgctttgcgattgctcacacaacgcttcgagtttacccgttctgcgattgatcgaaagatcaggacatcgacgggtgaactcgagttgggaagtgagcgatcgcaagcgatcgaaacggaaaactcgctgcttaccgtatgaataggaggtaccttctgccggtagtcgttcgttcagtaagctgagctcgaaagagctgtagtagttgaacggacgactaacttagatcgtagagaccgaggcatacggttccttgaaaaaggacgcaatgacctcggtttctacggtctaagtaaatcaatatcaccactactacctatgccacgaaaacccattgccgaggatccacaatggtgctcacgcgtttatgtagcattttgagcgggatcggttgagagaaatctcatggagttacgctcaagatgctagcacacgccgagcctatagagatggatcctgcttcgaaagaagctcctacggtctctatgggctcggtgtgtgcctagctcgtagctctaactccaatcatggtggaaaatgagtagtccatcgcagagtattcggcctgtgagcgttgttacggatttgctgcagcggatggagtttatgcgaaagcatagactctcgatcgcgcagcagatccgtattcccaaccacaggtcgaataccgatgtccggactgctcaaaaagagcggggttagcatgcgttgccatctcaacatctccgtactgcactctacatgacaagtacgagggtatcttgttcgtgagatcgttcatggtagcacgcagcttcggctgaggagcgatccacaacgctctagaaatagagctggtgacatcgctcttcagccgctcctaggtgctatcatgaacccttatgagaacaaaaagtcgcgtgggccccaatgcctagagctaaatgcgaaaggtgcaagctacgcacagcgtctgataaggcgagtgaaaactcgtcttagttcgtcttgtgcgtggcttgccgcgattccatttagttctaggtcgtctatcccatgcgacaaaaagatatcctccctctgaccatgtagcgtgcagtgcggagaagaggtgtgagacgcgcatgctgcgttgaaaaacgctcgaaaaccgtctcatacctctctccgtgatatcagtaggattcgtcagaggcgcatgaaaatgcggtacttgtgaatcctgctgatattacggagtgttgaggtggcaagttttcgaaacctcgctcccaccgtgataccgatccgagctatgagctagcataaatgcgtgagtaccattgccgtaggacggcgatgggttgcctcagacgcagccctagttatctacctttcgatccttggccacttcattggggacttcgaaagaagtatagacgaaagtggctaaggatgaatcgcgagataattagggctagacgaacggcaaaaaacgtggtatagcagcttacggtgatgttgatttccggcaggaggtacttcctatttcattgcgaagcggcgagaaaagctgtgcgcacgttgtgggggctactcaactagaagctgctgaaccgagccagcgatctcacgtaatctccc

SEQ ID NO: 13 - RNA, 6337 nt, synthetic construct
  source 1..6337: mol_type = other RNA
gggagaggatccaacatggagtgcggatatggttcgctaagggattccctgaatgcgaactctatcaactgtcgatacctggagacgatgctgatcgacctgtcatgggcgaaaacctataccgatgtaaactccgtatattcattttgctctagtccagtcctggaggttacttcggaaaaaagtaccgcagtggtgaagcgtgtcctccatacacctccgcaaggtattcacttttgtgatcatagttatgggtgtatgaggatatgcacttcactatgcagatgtgagatagatgtccgtgggcagatgtcagcgaaccgcgaagactcgcaatgaaaaaacgagtgaagggcgtcttggcgcgtccttgtctcacccaactggcttgtggttagagcttgactctgggatatgaccatcttggtcactaatttaggactgccctaacctccctaatggatgcgggtgataagttctgaatgtcacgtttgcaaatagcccttaatgttcccgtactgtggcacgagcaaaaaaccttacacctaaggcgatactcacttcaactgtgtgtatcacattaggtgcctacggtaaactcatcgtctagttctgggactgtttcgtctggttgaacgttataatagacacgatacctggttctaccattcgccgatccatttggtcttcgaaaaaacgagggagaatcactctatcaaagatgcacctcgtagcgagtgagtggaacttcataaagggaagtcatggccggtcagacttctggcactgatatgcaacatcagtacagtcttaagttccagccgaaagtgcggttggcatctcttaggacacagagcgattttggactggtagctgaccgcatgaaaaaaggaacgacgtgtcgaaaggtcccggtagtagctccctcattccacttggctaaacgttcaacacgtatcgagttggtttaggtagttcgcagacgcacaaacgaaggcaggtaaaacttggcaagttgcgtcgtggcacgtcataccagtgttgaaaaaacggctatgtagtgtctagctgtcaatacccgtacccatctgatggttgcaggatgattaggtcgaaacgaagtctctgatctgaggtcgtctgaagctaagtaatacctggctaacttgactaactcgtactcatactcagctttctcacattctgtgctcaaaatctgcattgactgcaacggtccaaaaaagcgaccttctgtgtgaatatgaatactaagcgggagttgaagaatagctcacagacagacacaacctacaaaatgaatgagcagtccgtgtaagctcgcattgctcacttcagccttcgggcgctatagccattattatgatccaactcgatcgaaaaaaggtactacgtagatttggccgacaccagattgcccgtaccgacaatgcggtttctttgtaaactgggcacttacgatcatagggagctggttacgaacggcatccgacaggaatctagctcgatgcatgggatagtactgtccacatccagccgtcccagagataggtagattgggaaaaaacgatcggtactgatctctggtgtctgacaaacacctccgcactcatttgagcatgagccaatgtataagttgcaccagaatcgctctggtatgtctaacatctgcaacatcttaagggcagtcatgactactgaccgtagtcggctagagcaccgtgaggccaaatgatcctccagaaaaaagcactgagttgacaccatccgagagtatggagcactagctatcatgacgaggttcccagttgaagtcagaatcttgatggacgaagcctactactacctgctgttggtacatggataagattggcttagtaggtcatccaagactgggccttggaaaaaaccacggtttgtgaccatgatcgtcccatgcatactgaaatcatcactagttgcggagtacgagtcgagctgtgcagtgcaaactaatccctttcggcggtcacatagtcctgaacgccgtccttatcaccgaaatcttccaacaaagcatggctcgtataggtgcccagtcgactactggatactggaaaaaacggactttagacagcaccctcaatctatgatcggtccagtggttagttcgtttctgcgagtttaccttgcatcaggatatgacacctcgggtgttgaagcctgaatagagagccggttcgatcttgtgtctactgaacgcagtgtagcgttagcaaaaaagacactatcctgaagcacgctatgttcgtaattcagccgactcgcattattgctggagcttcagctcggccttgactgagtgcactcaggcatatcagtcaacacagcaacttcctacgactgtcctaaatcaacactgctagtcacgtgtgtctatcgtctcgacctgcaagcatgggtgtcgtcgaaaaaagctcacgctgtacaaccttcaccccatagtgatagccacagaaaagcctctgaacaccaaccagacggtcgaaaagaaatgtaagctcactgcgtctggtgcgttgacaagaagacccattatgagcttacgtgctctcacgtaggcactatccaaaaaaggagtaaaggcgaacgttcgcagcagtttactcggtggtttatctctgaggtcacgtcgacctaagtcccatgatgacgtccagacaaccttcccttgcttccaaggctttggaggtatgctagagtcaagaattactctgcatcgagtcatcaagcattcagtactattagattggagcacgacacaaaaaagcatcttcaattaggcttatctgagacatctggtcaggtcaccgagtaccagatgtcggtagaaccaaagatgacataacagtgatcaaccgcaacttactgtaccctacacgagatatgtccgctatagcgtcaaacgcaggtactgcgatggaaaaaacagcagtagcacaggcttaacatcaatctggtggtcacctctatagggctagagtgacgggtatcggttatgacagtgttgcagtcagcaggtgcattgtcttcgtcgagcagtaagcggatagacaagggtcgacttggtctattatcatgtaacactccattacctggtctagaaatagaccaggtaccactacattacatgaagtcttcgcaagtcgacaggctataatccgcttcaaatggaacgaagacacgacttaagctgactgggtatgactcataaccgtgctgttgcactctaggttggatcaggtgaccagttacgctatgttaagcctgtgctactgctgaaaaaaccatcgcgcattgtccgtttgacatgcgataggacatatccaaccatcggtacagtcgtaatacgttgatcacccactcaccatctttgtacaggtagacatctggacaagccagacctgacgtaaacgttcagataagtagcgaacaagatgcaaaaaagtgtcgtgctccaatctaatagtagagtagacttgatgaccatctatcgagtaattcacagtgaaagcataccgtgtctatcttggaagctcaactcagttgtctgttacctgccatgggactactccatccgtgacctgctgaagtaaccaccgatgttgagtctgcgaacgttcgcctttactccaaaaaaggatagttatgatcggagagcacaccattgtataatgggtgatcagagcaacgcacgtacatatgtgagcttagtctgaccttcgaccgcactcgttgtgttcagaaagatggttgtggctaagcaaccagggtgaaggacagttgacgtgagcaaaaaacgacgacacccatgcttgcaggtccacagacaagacacactcctcatacagtgttgacgtcacgaagtcgtagtcccagaatgtgttgacaacggactctgagtgcctaaaccaaaggccgaggaaattggccagcaatctcattcatcggctgaagagacggtatagcgtgcttcaggatagtgtcaaaaaagctaacgattcctgtcgttcagtgctctttcgatcgaacctagccaggattcaggccgtgcttaccgaggtgtagactgtagatgcaagtatcgcaggcagaaacgtagggaggactggacctacgactcattgagggttgacaggtaagtccgaaaaaaccagtatccagtagtcgactgggctattgctggagccatggaatacctgaagatttccatatcgcggacggcgcctaatgttatgtgaccttgtatgaggattagtcaagtggacacagctcgttatcgcttccgcaacgctattctatttcagtactctttcaacgatcatggtcacaaaccgtggaaaaaaccaaggcatgtggacggatgaccatcacttgcaatcttatagaaagctcaacagcatccttatctaggcttcgagagatgcgattctgatcattggagggaacctcacgtgacaagctagtgagatgatttctcggatgtacggagttcagtgcaaaaaactggaggatcatttggcctcacggccaaggtaccgactactcaccactgtcatgactagtcaagggatgttgcgccttagggacataccacttggtacctggtgcatcgacacgattggctcacatgtgactgagtgcgcacacagatgtcagacagtcgtctacagtaccgatcgaaaaaacccaatctaccttagacgacgacggctggccagtcttagtactattgaaagagtcgagctagctacactgcggatgccaccgtctcccagctcccgcctacgttaagtgccactcaacaaaagaaaccagtacctgggtacgggagcgtaactgtcggccaaatctacgtagtaccaaaaaacgatcgaccctatagataataatgtatcgcatgcccgaagcagagatagagcaatgcacaatggtacggactgaatgcgagttttgtagggaaagagcgtctgtgatagtgatgtcaactccccaagtgatttcatattgaggtgttaggtcgcaaaaaaggaccgttgcagtcaatgcagatgtcacatgcagaatgtgccatgtacgagtatgaagcgataatagtcaagtggctctcttattacttccaatttcacgacctcacttcttgtacttcgttgatggagtatcatcctgtcgtgtagagatgggtcaacagcatgacagctagacactacatagccgaaaaaacaacactcaacactggtgccacgagtattacggccaagttgacgtcatcttcgtttgatatgtaccgaactacactcagtcactcgatactaagcacgcgtttagcttgcactgatgagggaggataaggagggaccttacttatactcgttccaaaaaacatgcggtcagctaccagtccaagtaccaagtgtgtcctatccatcaacaaccgcatcatacaatggaacttacatatcctctgatgttagtccgttgtgccagaacatttcttggccatgatgagttgatatgaagtttgttatgttcgctacgttaagtcgctttgatagagtgattctccctcgaaaaaacgaagacactgctcgtcggcgaattacctgtacaggtatctccaaagctataacgttaacgagtgcgaaacaggaagttgcctagacgatctgcgatacgtaggcattcaggacgatacacactccaatgatgagtatcagatgttatgtaaggaaaaaagctcgtgccacagtacgggaacaccttgacttatttgcaagtcatgatttcagaacgcgatatgcgcatccataactaaccttagggcatcgtgacgttagtgaccggcttttccatatcccttcactgtgctctaacctactcggtgttgggtgtatagcctacgcgccaagacgcccttcactcgaaaaaacattgcgtaatagaccggttcgctacgtttaccccacggatcgatgcatcacatctgtggttgctagtgcatagtgactagcacccataagagtcgtaacaaaagtctttgttgtgcggaggtaatcatctgacacgctggtcagtagcggtacaaaaaaccgaagtaacctccaggactggataccttggaaatgaatagtgtcaacttacatcgcagcaatatttcgcccagctgtctacgatcagctgtctgtgcaggtatcgttgtacagtagagttcgtctactcggaatccctcctaattgcatatccgtgtagtgggttggatcctctcgagctctccctttag

SEQ ID NO: 14 - DNA, 929 bp, synthetic construct
  source 1..929: mol_type = other DNA
gaattctaatacgactcactatagggagaggatccgaacactagccatagcagttcgctgagcgtaatgtgtatgaaacatcataagttcagtgctacattgaagcgaagagccaatgactcgttcgtgtcatactcatcaacggagtgttgactaagccgaaaaaacatagtccgactacacaccagacacgtttgaccctcagtcgattaactgcaagtcgcaaacaagctgacgtacagtaacgactcgtcactgtactgatgattccacaactgctaatgcacgaaaaaaggagtagtgtgtcagatcgacaagacttaaccacgattcctgatgcattgacttaccatcgactcaactgacaagggaccacgcagaggtgaatgagtcaggactttgtagtcggagtcggaaaaaacaccagtcacaatgtatcgtacgcttgctactaggagctcgtcatgacgttgagagcctgttaactagacacgttcctaagggttagccacacattaatatcgggcctgacacaggacacgaaaaaagaaggtgctgttagttggacaggtactatcatctcaagtcgatagtccaagtaggtttgaaccatgcatagcttgtatcaggtcatcgcctcaaacgttaggtgtcacattgtggaatcgcaaaaaacataccgacttccattatgggacacgtcgcttattcttggtaagtagaagttgccatcgtagtcgcacgacctacttatgacgaacttcggttaagtggctgacgtactaacagtgcgtgcaaaaaagacctacgaagccagagttcgttccagtgtgaaagtgcacatcacgagttgtgccaatgcacgttgcatcgagagttaatcccgtcttaagtagcaaggcacctgaatggaagttgattcgtctaga

SEQ ID NO: 15 - DNA, 939 bp, synthetic construct
  source 1..939: mol_type = other DNA
tctagaaatagacgaatcatgctgatctcaggtgctcacttgattaagacggctgtttatctcgatgccttcaatgttggcacaaatgcatcagtgcacttatgatagtgaacgaactctggcttcgtaggtcaaaaaagcacgcaagcatgtaacgtcagcctaacgcttgaagttcgcaggtgtgaggtcgtgccttgtttgtggcaactgtcatgacccaagaataagcgacgtgtcccatagatcagcacggtatgaaaaaagcgattcgtgaggtagacacctagatactctggcgatgacagtcattgagctatgcgagtcgataacctacttggactatcgacttgagtcacactgacctgtccatacatgctcaccttcaaaaaacgtgtccgcactatagcccgatatctcgtacaggctaacctcgttactcgtgtctagttaacaggctctcaactctacttagagctcctatcaagtgacgtacgattacctcacactggtgaaaaaaccgactcaagatttgaagtcctgtgagtatgacctctgcgtggtcccttgtcagttatggttcaggtaagtcactcgtgatggaatcgtaagcgttacttgtcgattatagtgcctactccaaaaaacgtgcatcttgatcagtggaatcatcagtacagtgacgagcttaggaagtacgtcaggactacgacgacttgcataaacaggactgagggagagtatcgtctggtgcaaatcttgactatgaaaaaacggcttagtcaacactccgttgaactcattcacacgaacgctgatacagctcttcgaacgtgcatagcactgacacacctgtgtttcattgtacgagcgctcagcgtgatcaagtggctagtgttcgctcgagctctctccctttagtgagggttaattaagctt

(19) United States
(12) Patent Application Publication
(10) Pub. No.: US 2025/0090567 A1
(43) Pub. Date: Mar. 20, 2025

(54) 5-FU AND RNAOG COMBINED IMMUNOCHEMOTHERAPY FOR PANCREATIC CANCER TREATMENT

(71) Applicant: ARIZONA BOARD OF REGENTS ON BEHALF OF ARIZONA STATE UNIVERSITY, Scottsdale, AZ (US)

(72) Inventors: Hao YAN, Chandler, AZ (US); Yang XU, Chandler, AZ (US); Xiaodong QI, Chandler, AZ (US); Guangbao YAO, Shanghai (CN); Chen GONG, Tempe, AZ (US); Yung CHANG, Tempe, AZ (US); Theresa YIP, Tempe, AZ (US)

(21) Appl. No.: 18/727,665

(22) PCT Filed: Jan. 10, 2023
(86) PCT No.: PCT/US2023/060370

§ 371 Date: Jul. 9, 2024

Related U.S. Application Data
(60) Provisional application No. 63/298,129, filed on Jan. 10, 2022.

(51) Int. Cl. A61K 31/7088; A61K 31/443; A61K 31/513; A61K 31/7072; A61K 45/06; A61P 35/00

(52) U.S. Cl. CPC A61K 31/7088; A61K 31/443; A61K 31/513; A61K 31/7072; A61K 45/06; A61P 35/00

(57) ABSTRACT

Described herein are compositions comprising RNA origami (RNAOG) which include 5-fluorouracil (5FU) or analogues thereof are described which can be used to both directly treat a cancer by inhibiting a biochemical pathway overexpressed in a cancer cell and indirectly treat a cancer by inducing an anti-cancer immune response. Also described herein are methods of using said compositions for treating cancer, inducing an anti-cancer immune response, and/or killing a cancer cell.

### RELATED APPLICATIONS

This disclosure claims priority to U.S. Provisional Application No. 63/298,129, filed Jan. 10, 2022, the contents of which are herein incorporated by reference in their entirety.

### FIELD

This disclosure relates to compositions comprising RNA origami (RNAOG) and 5-fluorouracil (5FU) or analogues thereof. Also described herein are methods of treating cancer using said RNAOG and 5FU or 5FU analogue compositions.

### SEQUENCE STATEMENT

The instant application contains a Sequence Listing, which has been submitted electronically and is hereby incorporated by reference in its entirety. Said file, is named G8118-03501 SEQ ID LISTING.xml and is 46 kb in size.

### BACKGROUND

Single stranded RNA (ssRNA) and double stranded RNA (dsRNA) can be detected by pattern recognition receptors in mammalian cells and synthetic ssRNA and dsRNA have been explored as immuno-stimulating adjuvants (Alexopoulou, et al., 2001. Nature 413:732-738.). For example, polyinosinic:polycytidylic acid (polyIC), a synthetic analog of dsRNA, has been widely studied as an adjuvant in treating diseases such as upper respiratory tract infections and tumors, therefore, allowing it to be explored as an adjuvant in flu and cancer vaccines. However, susceptibility of dsRNA to nuclease digestion tends to be a concern especially when they are used in vivo.

Pancreatic ductal adenocarcinoma (PDA) is one of the most immune-resistant tumor types. Single-agent immune modulators targeting immune checkpoint blockade and multi-modal therapies including target immunotherapy have been proven to be clinically ineffective.

Recently, nucleic acid nanotechnology has emerged as a promising approach for cancer targeting and treatment.

### SUMMARY

As described in this disclosure, the present invention provides for a composition comprising RNA origami nanostructure (RNAOG) and an anti-cancer therapy for the treatment of cancer.

Previous disclosures have noted the ability of RNA origami to act as a TLR3—specific agonist (Qi et al., ACS Nano 2020, 14, 4, 4727-4740). RNA origami nanostructures which can be used in anti-cancer compositions and treatments include those described in US20210246452, incorporated by reference.

In some aspects, the anti-cancer therapy includes 5-fluorouracil (5-FU).

In some aspects, this disclosure provides for a method of inducing an immune response a subject, comprising administering to the subject an effective amount of a RNAOG and 5FU or a 5FU analogue.

In some aspects, this disclosure provides for a method of treating a disease or disorder in a subject, comprising administering to the subject in need thereof a therapeutically effective amount of a RNAOG and 5FU or a 5FU analogue. The disease or disorder can be cancer. In some aspects, the cancer is pancreatic cancer. In some aspects, the 5FU analogue is selected from capetabine, UFT, tegafur, carmofur, U-332 or floxuridine.

In some aspects, the method, further comprises administering at least one therapeutic agent to the subject. In some aspects, therapeutic agent is a chemotherapeutic drug. In some aspects, the chemotherapeutic drug is selected from:, Abraxane (Paclitaxel Albumin-stabilized Nanoparticle Formulation), Afinitor (Everolimus), Erlotinib Hydrochloride, Everolimus, Gemcitabine Hydrochloride, Irinotecan Hydrochloride, Lynparza (Olaparib), Mitomycin, Olaparib, cyclophosphamide, doxorubicin, oxaliplatin, mitoxantrone, or Sunitinib Malate.

In some aspects, the 5FU or 5FU analogue is covalently connected to the RNAOG sequence. The covalent connection between the 5FU or 5FU analogue and the RNAOG is a phosphodiester bond. In some aspects, the 5FU or 5FU analogue is not covalently connected to the RNAOG sequence.

In some aspects, this disclosure provides for a method of reducing the proliferation of a cancer tumor cell, the method comprising contacting said cancer tumor cell with a RNAOG and 5FU or a 5FU analogue. In some aspects, the 5FU or 5FU analogue is covalently connected to the RNAOG sequence. The covalent connection between the 5FU or 5FU analogue and the RNAOG is a phosphodiester bond. In some aspects, the 5FU or 5FU analogue is not covalently connected to the RNAOG sequence. In some aspects, the 5FU analogue is selected from capetabine, UFT, tegafur, carmofur, U-332 or floxuridine.

In some aspects, this disclosure provides for a use of a RNAOG and 5FU or a 5FU analogue for the manufacture of a medicament for inducing an immune response in a subject.

In some aspects, this disclosure provides for a composition comprising RNAOG, 5FU, and a pharmaceutically acceptable carrier. In some aspects, the RNAOG is covalently linked to 5FU. In some aspects, the composition further comprises a chemotherapeutic drug. In some aspects, the chemotherapeutic drug is selected from: doxorubicin, Abraxane (Paclitaxel Albumin-stabilized Nanoparticle Formulation), Afinitor (Everolimus), Erlotinib Hydrochloride, Everolimus, Gemcitabine Hydrochloride, Irinotecan Hydrochloride, Lynparza (Olaparib), Mitomycin, Olaparib, and Sunitinib Malate. In some aspects, the composition further comprises an irreversible dihydropyrimidine dehydrogenase (DPD) inactivator.

In some aspects, this disclosure provides for the use of a composition comprising RNAOG covalently linked to 5FU for the manufacture of a medicament for treating a disease or disorder in a subject.

In some aspects, this disclosure provides for a composition comprising RNAOG covalently linked to 5FU for the prophylactic or therapeutic treatment of a disease or disorder in a subject.

In some aspects, this disclosure provides for a kit comprising a composition comprising RNAOG covalently linked to 5FU and instructions for administering the composition to a subject to induce an immune response or to treat a disease or disorder. The kit can further comprise at least one therapeutic agent. The therapeutic agent can be selected from: doxorubicin, Abraxane (Paclitaxel Albumin-stabilized Nanoparticle Formulation), Afinitor (Everolimus), Erlotinib Hydrochloride, Everolimus, Gemcitabine Hydrochloride, Irinotecan Hydrochloride, Lynparza (Olaparib), Mitomycin, Olaparib, and Sunitinib Malate.

In some aspects, the RNAOG comprises a nanostructure which comprises an NA (nucleic acid) sequence of about 1000 to about 10,000 nucleotides in length. In some embodiments, the NA sequence is about 1800 to about 7500 nucleotides in length. In some aspects, the RNAOG comprises a nucleic acid sequence having at least about 75, 80, 85, 90, 95, 97, or 99%, or 100% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 or SEQ ID NO:13. Sequences are described in the included Sequence ID listing provided with this disclosure.

### BRIEF DESCRIPTION OF DRAWINGS

FIGS. 1A-C. 5FU-RNAOG synthesis and characterization.

FIG. 1A. 5F-UTP was directly incorporated into the RNAOG molecules in the in vitro transcription reaction.

FIG. 1B. AFM images of RNAOG and 5FU-RNAOG. Scale bars represent 100 nm.

FIG. 1C. RNAOG (top) and 5FU-RNAOG (bottom) was incubated in 10% FBS at 37° C. and analyzed by agarose gel electrophoresis at various time points.

FIG. 2. Pancreatic cancer cell growth inhibition assay. The MiaPaca-2 cells were seeded in the 96-well plates. Various doses of 5FU-RNAOG and 5FU were introduced and the cells were incubated for 3, 4, or 5 days. The cell viability was quantitated by cell counting kit-8 following the manufacturer's instruction.

FIG. 3A-B. MiaPaca-2 cell line colony formation assay.

FIG. 3A. Images of MiaPaca-2 cell colonies with different treatments as indicated. Triplicates were performed and shown for each treatment.

FIG. 3. Area of colony was quantitated for each drug treatment.

FIG. 4A-D. Anti-tumor activity of RNAOG in combination with 5FU.

FIG. 4A. schematic illustration of tumor inoculation and treatment schedule. Nude mice received subcutaneous (SC) injection of 3×106 MiaPaca2-iRFP on day 0. Starting on week 4, the mice received intratumor injections of PBS, 5FU, RNAOG, RNAOG+5FU, or 5FU-RNAOG twice a week for total 7 times.

FIG. 4B. Tumor progression monitored by LI-COR Imaging of near infrared fluorescence intensity from the MiaPaca2-iRFP cell line that expresses iRFP.

FIG. 4C. Tumor dimensions were also measured for each treatment.

FIG. 4D. The survival curve of each treatment group. **and***indicate p<0.01 and p<0.001, respectively.

FIG. 5. Immunohistochemical analysis of Arginase, F4/80, Ki67 expression in xenograft tumor tissue. Expression of Arg1, F4/80, Ki67 were analyzed using xenograft tumor tissue. The tumor tissues were stained with Arg1, F4/80 and Ki67 monoclonal antibodies Representative microscopy images of tumor section stained with F4/80 and Arg1 antibodies for mouse macrophages and M2-like TAM. The brown color (darker shading) represents positive result. Average expression of F4/80 and Arg1 quantified in the 3 tumor sections. Representative microscopy images of tumor section stained with ki67 antibody for cell nuclei (dark brown-darker shading)). Average nuclear expression of Ki67 quantified in the 3 tumor sessions.

FIG. 6A-C. Colony formation assay of MiaPaca-2 and RAW264.7 cells in 2D culture. The MiaPaca-2 cells were cultured with the addition of PBS, 5FU+RAW264.7 (5FU/Mac), RNAOG+RAW264.7 (RG/Mac), or 5FU+RNAOG+RAW264.7 (5FU+RG/Mac). FIG. 6A. The general scheme for the culturing. The colony assay was performed as described in FIG. 3. FIG. 6B. Images of the colonies of the treated cohorts: PBS (left column), 5FU/Mac (second column from left), RG/Mac (third column from left), 5FU+RG/Mac (rightmost column). FIG. 6C. Quantitation of the colonies for each treated cohort.

FIG. 7A-B. Clustering display of M1-like TAM associated genes after RNAOG treatment in RAW264.7 cells by RT-qPCR analysis. RNA was extracted using RNA isolation kit for gene expression analysis after RNAOG treatment for 6 hours in RAW264.7 cells. The mRNA levels of M1-like TAM associated cytokine and chemokine genes in RAW264.7 were determined using real-time RT-PCR. Data represent the ratio of relative mRNA levels versus PBS untreated control cells. FIG. 7A. Clustering display information for II6, INOS, TNFa, II12b, II1a, and II1b genes. FIG. 7B. Clustering display information for II1a, ccl5, Rps29, ccl22, and ccl3 genes.

FIG. 8. RNAOG internalization analysis. RAW264.7 and MiaPaca-2 cells were cocultured and incubated with AF488 labeled RNAOG. Confocal images were shown with 4 hours (top) and 16 hours (bottom) incubation. Green fluorescence indicates the RNAOG-AF488, and red color represents the endosomes/lysosomes as stained by lysotracker red dye. The purple-colored cells are MiaPaca2-iRFP with iRFP signals.

FIG. 9. Cell viability analysis of cocultured RAW 264.7 and MiaPaca-2 cells through flow cytometry. The RAW 264.7 and MiaPaca-2 cells were harvested and separated in the flow cytometry by CD11b+iRFP- and CD11b−iRFP+, respectively. The live and dead cells were quantitated by zombie yellow fluorescence. 80.5%, 92.6%, and 75.1% of the RAW 264.7 cells were live cells after 24, 48, and 72 hours treated with PBS. While only 41.8%, 10.8% and 13.0% of the RAW 264.7 cells were live cells after 24, 48, and 72 hours treated with RNAOG. About half of the MiaPaca2 cells survived in the coculture after 72 hours in the presence or absence of RNAOG.

### DETAILED DESCRIPTION

The entire document is intended to be related as a unified disclosure, and it should be understood that all combinations of features described herein are contemplated, even if the combination of features are not found together in the same sentence, or paragraph, or section of this document. The present disclosure as illustratively described in the following may suitably be practiced in the absence of any element or elements, limitation or limitations, not specifically disclosed herein.

### Certain Definitions

Technical terms are used by their common sense unless indicated otherwise. If a specific meaning is conveyed to certain terms, definitions of terms will be given in the following in the context of which the terms are used.

The singular forms “a”, “an”, and “the” may refer to plural articles unless specifically stated otherwise.

As used herein, the term “about” means ±10%.

As used herein, the term “operably-linked” refers to the association two chemical moieties so that the function of one is affected by the other, e.g., an arrangement of elements wherein the components so described are configured so as to perform their usual function.

As used herein, the term “nucleic acid” refers to deoxyribonucleotides or ribonucleotides and polymers thereof in either single- or double-stranded form, comprising monomers (nucleotides) containing a sugar, phosphate and a base that is either a purine or pyrimidine. Unless specifically limited, the term encompasses nucleic acids containing analogues of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

As used herein, the terms “nucleotide sequence” and “nucleic acid sequence” refer to a sequence of bases (purines and/or pyrimidines) in a polymer of DNA or RNA, which can be single-stranded or double-stranded. In some embodiments, the nucleotide sequence comprises synthetic, non-natural or altered nucleotide bases, and/or backbone modifications (e.g., a modified oligomer, which can include or exclude a morpholino oligomer, phosphorodiamate morpholino oligomer or vivo-mopholino). The terms “oligo”, “oligonucleotide” and “oligomer” may be used interchangeably and refer to such sequences of purines and/or pyrimidines. The terms “modified oligos”, “modified oligonucleotides” or “modified oligomers” may be similarly used interchangeably, and refer to such sequences that contain synthetic, non-natural or altered bases and/or backbone modifications (e.g., chemical modifications to the internucleotide phosphate linkages and/or to the backbone sugar).

Modified nucleotides can include or exclude alkylated purines; alkylated pyrimidines; acylated purines; and acylated pyrimidines. These classes of pyrimidines and purines can include or exclude pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine. Backbone modifications can include or exclude chemical modifications to the phosphate linkage. The chemical modifications to the phosphate linkage can include or exclude e.g. phosphorodiamidate, phosphorothioate (PS), N3′phosphoramidate (NP), boranophosphate, 2′,5′phosphodiester, amide-linked, phosphonoacetate (PACE), morpholino, peptide nucleic acid (PNA), inverted linkages (5′-5′ and 3′-3′ linkages)) and sugar modifications (e.g., 2′-O-Me, UNA, LNA).

The oligonucleotides described herein may be synthesized using solid or solution phase synthesis methods. In some embodiments, the oligonucleotides are synthesized using solid-phase phosphoramidite chemistry (U.S. Pat. No. 6,773,885, herein incorporated by reference) with automated synthesizers, herein incorporated by reference. Chemical synthesis of nucleic acids allows for the production of various forms of the nucleic acids with modified linkages, chimeric compositions, and nonstandard bases or modifying groups attached in chosen places through the nucleic acid's entire length. In some embodiments, the oligonucleotides described herein may be synthesized using enzymatic methods which can include adding single-bases via an enzyme.

Some embodiments of the invention encompass isolated or substantially purified nucleic acid compositions. As used herein an “isolated” or “purified” DNA molecule or RNA molecule refers to a DNA molecule or RNA molecule that exists apart from its native environment and is therefore not a product of nature. An isolated DNA molecule or RNA molecule may exist in a purified form or may exist in a non-native environment. In some embodiments, the non-native environment can include or exclude a transgenic host cell. In some embodiments, the terms “isolated” or “purified” includes a nucleic acid molecule which is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

By “portion” or “fragment,” as it relates to a nucleic acid molecule, sequence or segment of the invention, when it is linked to other sequences for expression, is meant a sequence having at least 80 nucleotides, at least 150 nucleotides, or at least 400 nucleotides. If not employed for expressing, a “portion” or “fragment” means at least 9, at least 12, at least 15, or at least 20, consecutive nucleotides, e.g., probes and primers (oligonucleotides), corresponding to the nucleotide sequence of the nucleic acid molecules of the invention.

“Recombinant DNA molecule” is a combination of DNA sequences that are joined together using recombinant DNA technology and procedures to join together DNA sequences as described in Sambrook and Russell, Molecular Cloning: A Laboratory Manual, Cold Spring Harbor, NY: Cold Spring Harbor Laboratory Press (3rd edition, 2001), herein incorporated by reference.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

The following terms are used to describe the sequence relationships between two or more nucleotide sequences: (a) “reference sequence,” (b) “comparison window,” (c) “sequence identity” (d) “percentage of sequence identity,” (e) “substantial identity” and (f) “complementarity”.

(a) As used herein, “reference sequence” is a defined sequence used as a basis for sequence comparison. A reference sequence may be a subset or the entirety of a specified sequence. In some embodiments, the specified sequence can include or exclude a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally is 30 contiguous nucleotides, 40 contiguous nucleotides, 50 contiguous nucleotides, 100 contiguous nucleotides, or longer. Those of skill in the art understand that to avoid a high similarity to a reference sequence due to inclusion of gaps in the polynucleotide sequence a gap penalty is typically introduced and is subtracted from the number of matches.

In some embodiments, methods of alignment of sequences for comparison are determined by mathematical algorithm. In some embodiments, the determination of percent identity, including sequence complementarity, between any two sequences is accomplished using a mathematical algorithm. In some embodiments, such mathematical algorithms can include or exclude the algorithm of Myers and Miller (Myers and Miller, CABIOS, 4, 11 (1988)); the local homology algorithm of Smith et al. (Smith et al., Adv. Appl. Math., 2, 482 (1981)); the homology alignment algorithm of Needleman and Wunsch (Needleman and Wunsch, JMB, 48, 443 (1970)); the search-for-similarity-method of Pearson and Lipman (Pearson and Lipman, Proc. Natl. Acad. Sci. USA, 85, 2444 (1988)); the algorithm of Karlin and Altschul (Karlin and Altschul, Proc. Natl. Acad. Sci. USA, 87, 2264 (1990)), modified as in Karlin and Altschul (Karlin and Altschul, Proc. Natl. Acad. Sci. USA 90, 5873 (1993), all of which are herein incorporated by reference.

In some embodiments, computer implementations of these mathematical algorithms are utilized for comparison of sequences to determine sequence identity or complementarity. Such implementations can include or exclude: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (Higgins et al., CABIOS, 5, 151 (1989)); Corpet et al. (Corpet et al., Nucl. Acids Res., 16, 10881 (1988)); Huang et al. (Huang et al., CABIOS, 8, 155 (1992)); and Pearson et al. (Pearson et al., Meth. Mol. Biol., 24, 307 (1994)). The ALIGN program is based on the algorithm of Myers and Miller, supra. The BLAST programs of Altschul et al. (Altschul et al., JMB, 215, 403 (1990)) are based on the algorithm of Karlin and Altschul supra.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. A test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. Alignment may also be performed manually by inspection.

Comparison of nucleotide sequences for determination of percent sequence identity may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the program.

(c) As used herein, the terms “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. In some embodiments, the identity between any two nucletic acid sequences is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, 95%, 96%, 97%, 98%, or 99%.

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even at least 95%, 96%, 97%, 98%, or 99% sequence identity, compared to a reference sequence using one of the alignment programs described herein using standard parameters.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Another indication that nucleotide sequences are substantially identical is if two molecules hybridize to each other under stringent conditions. Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. As used herein, the term “bind(s) substantially” refers to complementary hybridization between a probe nucleic acid and a target nucleic acid and embraces minor mismatches that in some embodiments is accommodated by reducing the stringency of the hybridization media to achieve the desired detection of the target nucleic acid sequence.

(f) The term “complementary” as used herein refers to the broad concept of complementary base pairing between two nucleic acids aligned in an antisense position in relation to each other. When a nucleotide position in both of the molecules is occupied by nucleotides normally capable of base pairing with each other, then the nucleic acids are considered to be complementary to each other at this position. Two nucleic acids are substantially complementary to each other when at least about 50%, at least about 60%, or at least about 80% of corresponding positions in each of the molecules are occupied by nucleotides which normally base pair with each other (e.g., A:T (A:U for RNA) and G:C nucleotide pairs).

As used herein, the term “derived” or “directed to” with respect to a nucleotide molecule means that the molecule has complementary sequence identity to a particular molecule of interest.

The term “subject” as used herein refers to humans, higher non-human primates, rodents, domestic, cows, horses, pigs, sheep, dogs and cats. In one embodiment, the subject is a human.

The term “therapeutically effective amount,” in reference to treating a disease state/condition, refers to an amount of a therapeutic agent that is capable of having any detectable, positive effect on any symptom, aspect, or characteristics of a disease state/condition when administered as a single dose or in multiple doses. Such effect need not be absolute to be beneficial.

The terms “treat’ and “treatment” refer to both therapeutic treatment and prophylactic or preventative measures, wherein the object is to prevent or decrease an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The terms “inhibiting” or “reducing” or any variation of these terms includes any measurable decrease or complete inhibition to achieve a desired result. The terms “promote” or “increase” or any variation of these terms includes any measurable increase or production of a protein or molecule to achieve a desired result.

The term “preventing” or any variation of this term means to slow, stop, or reverse progression toward a result. The prevention may be any slowing of the progression toward the result.

As used herein, the term “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

The tumor microenvironment is an important aspect of cancer biology that contributes to tumor initiation, tumor progression and responses to therapy. The tumor microenvironment is composed of a heterogeneous cell population that includes malignant cells and cells that support tumor proliferation, invasion, and metastatic potential though extensive crosstalk. Tumor cells often induce an immunosuppressive microenvironment, which favors the development of immunosuppressive populations of immune cells, such as myeloid-derived suppressor cells (MDSCs), tumor-associated macrophage (TAM), and regulatory T cells (Tregs). Therefore, targets within the tumor microenvironment have been uncovered that can help direct and improve the actions of various cancer therapies, notably immunotherapies that work by potentiating host antitumor immune responses.

The present invention surprisingly found that a combination of a RNAOG and 5FU or a 5FU analogue significantly improves immune response, regulates tumor microenvironment and therefore dramatically improves anti-cancer activity.

### Nucleic Acid Nanostructures as the Basis of RNA Origami (RNAOG)

In some embodiments, this disclosure provides for a NA nanostructure comprising a nucleic acid sequence having at least about 60% sequence identity to any one of SEQ ID NO:1-13. In some embodiments, the NA nanostructure comprises a nucleic acid sequence having at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO:1-13. In some embodiments, the NA nanostructure consists of a nucleic acid sequence having at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO:1-13. In certain embodiments, the NA nanostructure comprises any sequence comprising any one of SEQ ID NO: 1-13. In some embodiments, the NA nanostructure consists of any one of SEQ ID NO:1-13. In some embodiments, the NA nanostructure further comprises a 5-FU or 5-FU analogue within or at the terminae (5′ or 3′) of the sequence.

In some embodiments, the NA nanostructure comprises one or more modified nucleic acids. In some embodiments, the one or more modified nucleic acids are selected from inosine residues, alkynyl-modified nucleotides,

In some embodiments, the alkynyl modified nucleotides are chemically synthesized from a phosphoramidite selected from: 5′-Dimethoxytrityl-5-[(6-oxo-6-(dibenzo[b,f]azacyclooct-4-yn-1-yl)-capramido-N-hex-6-yl)-3-acrylimido]-2′-deoxyUridine,3′-[(2-cyanoethyl)-(N,N-diisopropyl)]-phosphoramidite, 5′-Dimethoxytrityl-5-ethynyl-2′-deoxyUridine, 3′-[(2-cyanoethyl)-(N,N-diisopropyl)]-phosphoramidite, 5′-Hexynyl Phosphoramidite, 5′-Dimethoxytrityl-5-(octa-1,7-diynyl)-2′-deoxyuridine, 3′-[(2-cyanoethyl)-(N,N-diisopropyl)]-phosphoramidite, 10-(6-oxo-6-(dibenzo[b,f]azacyclooct-4-yn-1-yl)-capramido-N-ethyl)-O-tricthyleneglycol-1-[(2-cyanoethyl)-(N,N-diisopropyl)]-phosphoramidite, 6-Bromo-hex-1-yl-(2-cyanoethyl)-(N,N-diisopropyl)-phosphoramidite, 3-Dimethoxytrityloxy-2-(3-(5-hexynamido)propanamido)propyl-1-O-[(2-cyanoethyl)-(N,N-diisopropyl)]-phosphoramidite, and 5′-Dimethoxytrityl-3′-propargyl-5-methyl-2′-deoxyCytosine-N-succinoyl-long chain alkylamino-CPG.

In some embodiments, one or more agents (e.g., 1, 2, 3, 4, 5, 6, 7, 8, 9, 10 etc.) are operably linked to the NA nanostructure. The one or more agents can include or exclude diagnostic agents or therapeutic agents. In some embodiments, at least one diagnostic agent is operably linked to the NA nanostructure. In some embodiments, at least one therapeutic agent is operably linked to the NA nanostructure. In some embodiments, at least one diagnostic agent and at least one therapeutic agent are operably linked to the NA nanostructure.

Diagnostic agents can include or exclude fluorophores, radioisotopes, nanoparticles, and colorimetric indicators.

As used herein, the term “therapeutic agent” refers to agents that provide a therapeutically desirable effect when administered to an animal. The animal is a mammal, which can include or exclude a human. The therapeutic agent may be of natural or synthetic origin. In some embodiments, the therapeutic agent can include or exclude a nucleic acid, a polypeptide, a protein, a peptide, a radioisotope, saccharide or polysaccharide or an organic compound, which can include or exclude a small molecule. The term “small molecule” includes organic molecules having a molecular weight of less than about, e.g., 1000 daltons. In one embodiment a small molecule can have a molecular weight of less than about 800 daltons. In another embodiment a small molecule can have a molecular weight of less than about 500 daltons.

### Certain Methods

In some embodiments, an NA nanostructure described herein is used as an immuno-adjuvant to boost an immune response. In some embodiments, the immune response induces anti-tumor immunity.

Certain embodiments of the invention provide a method of inducing an immune response a subject. The subject is a mammal, which can include or exclude a human. The method comprises administering to the subject an effective amount of a composition as described herein.

In some embodiments, the administration of the composition described herein increases an immune response by at least about, e.g., 20%, 30%, 40%, 50%, 60%, 70%, 80%, 90%, or more, compared to a control. Methods of measuring an immune response include using an assay as described in the Examples. As used herein, the phrase “inducing an immune response” refers to the activation of an immune cell. Methods of measuring an immune response include using an assay described in the Examples. As used herein, the phrase “effective amount” refers to an amount of a composition described herein that induces an immune response.

Certain embodiments of the invention also provide a method of treating a disease or disorder in a subject, comprising administering to the subject a therapeutically effective amount of a composition as described herein.

In some embodiments, a method of the invention further comprises administering at least one therapeutic agent to the subject. In some embodiments, the at least one therapeutic agent is administered in combination with the NA nanostructure. As used herein, the phrase “in combination” refers to the simultaneous or sequential administration of the NA nanostructure and the at least one therapeutic agent. For simultaneous administration, the NA nanostructure and the at least one therapeutic agent is present in a single composition or is separate. In some embodiments, when the NA nanostructure and at least one thereapeutic agent are administered simultaneously, they are administered by either the same or different routes.

In some embodiments, this disclosure provides a method of inducing an immune response a subject (e.g., a mammal, which can include or exclude a human), comprising administering to the subject an effective amount of a RNAOG with a 5FU or 5FU analogue.

In some embodiments, this disclosure provides a method of treating a disease or disorder in a subject, comprising administering to the subject a therapeutically effective amount of a nanostructure or a composition as described herein.

In some embodiments, the disease or disorder is cancer.

In some embodiments, the cancer is pancreatic cancer.

In some embodiments, the method further comprises administering at least one therapeutic agent to the subject.

In some embodiments, the 5FU is covalently attached to RNAOG (RNA-origami).

In some embodiments, the therapeutic agent is a chemotherapeutic drug. In some embodiments, the chemotherapeutic drug is selected from: Abraxane (chemical name: albumin-bound or nab-paclitaxel), Adriamycin (chemical name: doxorubicin), carboplatin (brand name: Paraplatin), Cytoxan (chemical name: cyclophosphamide), daunorubicin (brand names: Cerubidine, DaunoXome), Doxil (chemical name: doxorubicin), Ellence (chemical name: epirubicin), fluorouracil (also called 5-fluorouracil or 5-FU; brand name: Adrucil), Gemzar (chemical name: gemcitabine), Halaven (chemical name: eribulin), Ixempra (chemical name: ixabepilone), methotrexate (brand names: Amethopterin, Mexate, Folex), Mitomycin (chemical name: mutamycin), mitoxantrone (brand name: Novantrone), Navelbine (chemical name: vinorelbine), Taxol (chemical name: paclitaxel), Taxotere (chemical name: docetaxel), thiotepa (brand name: Thioplex), vincristine (brand names: Oncovin, Vincasar PES, Vincrex), and Xeloda (chemical name: capecitabine). In some embodiments, the chemotherapeutic agent is selected from: Abraxane (Paclitaxel (with albumin) Injection), Adriamycin (Doxorubicin), Afinitor (Everolimus), Alecensa (Alectinib), Alimta (PEMETREXED), Aliqopa (Copanlisib), Alkeran Injection (Melphalan), Alunbrig (Brigatinib), Aredia (Pamidronate), Arimidex (Anastrozole), Aromasin (Exemestane), Arranon (Nelarabine), Arzerra (Ofatumumab), Avastin (Bevacizumab), Bavencio (Avelumab), Beleodaq (Belinostat), Besponsa (Inotuzumab Ozogamicin), Bexxar (Tositumomab), BiCNU (Carmustine), Blenoxane (Bleomycin), Blincyto (Blinatumomab), Bosulif (Bosutinib), Braftovi (Encorafenib), Busulfex (Busulfan), Cabometyx (Cabozantinib), Calquence (Acalabrutinib), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Cinqair (Reslizumab), Clolar (Clofarabine), Cometriq (Cabozantinib), Copiktra (Duvelisib), Cosmegen (Dactinomycin), Cotellic (Cobimetinib), Cyramza (Ramucirumab), CytosarU (Cytarabine), Cytoxan (Cytoxan), Cyclophosphamide, Dacogen (Decitabine), Darzalex (Daratumumab), DaunoXome (Daunorubicin Lipid Complex), Daurismo (Glasdegib), Decadron (Dexamethasone), DepoCyt (Cytarabine Lipid Complex), Dexamethasone Intensol (Dexamethasone), Dexpak Taperpak (Dexamethasone), Docefrez (Docetaxel), Doxil (Doxorubicin Lipid Complex), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Emend (Fosaprepitant), Empliciti (Elotzumab), Erbitux (Cetuximab), Erivedge (Vismodegib), Erleada (Apalutamide), Erwinaze (Asparaginase Erwinia chrysanthemi), Ethyol (Amifostine), Etopophos (Etoposide), Eulexin (Flutamide), Fareston (Toremifene), Farydak (Panobinostat), Faslodex (Fulvestrant), Femara (Letrozole), Firmagon (Degarelix), FloPred (Prednisolone), Fludara (Fludarabine), Folex (Methotrexate), Folotyn (Pralatrexate), FUDR (FUDR (floxuridine)), Gazyva (Obinutuzumab), Gemzar (Gemcitabine), Gilotrif (Afatinib), Gleevec (Imatinib Mesylate), Halaven (Eribulin), Herceptin (Trastuzumab), Hexalen (Altretamine), Hycamtin (Topotecan), Hycamtin (Topotecan), Hydrea (Hydroxyurea), Ibrance (Palbociclib), Iclusig (Ponatinib), Idamycin PFS (Idarubicin), Idhifa (Enasidenib), Ifex (Ifosfamide), Imbruvica (Ibrutinib), Imfinzi (Durvalumab), Imlygic (Talimogene Laherparepvec), Inlyta (Axitinib), Intron A alfab (Interferon alfa-2a), Iressa (Gefitinib), Istodax (Romidepsin), Ixempra (Ixabepilone), Jakafi (Ruxolitinib), Jevtana (Cabazitaxel), Kadcyla (Ado-trastuzumab Emtansine), Keytruda (Pembrolizumab), Kisqali (Ribociclib), Kyprolis (Carfilzomib), Lanvima (Lenvatinib), Leukeran (Chlorambucil), Leukine (Sargramostim), Leustatin (Cladribine), Lorbrena (Lorlatinib), Lupron (Leuprolide), Lynparza (Olaparib), Lysodren (Mitotane), Matulane (Procarbazine), Megace (Megestrol), Mekinist (Trametinib), Mektovi (Binimetinib), Mesnex (Mesna), Mustargen (Mechlorethamine), Mutamycin (Mitomycin), Myleran (Busulfan), Mylotarg (Gemtuzumab Ozogamicin), Navelbine (Vinorelbine), Nerlynx (Neratinib), Neulasta (filgrastim), Neulasta (pegfilgrastim), Neupogen (filgrastim), Nexavar (Sorafenib), Nilandron (Nilandron (nilutamide)), Ninlaro (Ixazomib), Nipent (Pentostatin), Nolvadex (Tamoxifen), Odomzo (Sonidegib), Oncaspar (Pegaspargase), Oncovin (Vincristine), Opdivo (Nivolumab), Panretin (Alitretinoin), Paraplatin (Carboplatin), Perjeta (Pertuzumab), Platinol (Cisplatin), PlatinolAQ (Cisplatin), Pomalyst (Pomalidomide), Portrazza (Necitumumab), Proleukin (Aldesleukin), Purinethol (Mercaptopurine), Reclast (Zoledronic acid), Revlimid (Lenalidomide), Rituxan (Rituximab), RoferonA alfaa (Interferon alfa-2a), Rubex (Doxorubicin), Rubraca (Rucaparib), Rydapt (Midostaurin), Sandostatin (Octreotide), Soltamox (Tamoxifen), Sprycel (Dasatinib), Stivarga (Regorafenib), Sutent (Sunitinib), Sylvant (Siltuximab), Synribo (Omacetaxine), Tabloid (Thioguanine), Taflinar (Dabrafenib), Tagrisso (Osimertinib), Talzenna (Talazoparib), Tarceva (Erlotinib), Targretin Capsules (Bexarotene), Tasigna (Decarbazine), Taxol (Paclitaxel), Taxotere (Docetaxel), Tecentriq (Atezolizumab), Temodar (Temozolomide), Tepadina (Thiotepa), Thioplex (Thiotepa), Tibsovo (Ivosidenib), Toposar (Etoposide), Torisel (Temsirolimus), Treanda (Bendamustine hydrochloride), Trelstar (Triptorelin), Tykerb (lapatinib), Unituxin (Dinutuximab), Valstar (Valrubicin), Varubi (Rolapitant), Vectibix (Panitumumab), Velban (Vinblastine), Velcade (Bortezomib), Venclexta (Venetoclax), Vepesid (Etoposide), Vepesid (Etoposide Injection), Verzenio (Abemaciclib), Vesanoid (Tretinoin), Vidaza (Azacitidine), Vincasar PFS (Vincristine), Vincrex (Vincristine), Vistogard (Uridine Triacetate), Vitrakvil (Larotrectinib), Vizimpro (Dacomitinib), Votrient (Pazopanib), Vumon (Teniposide), Wellcovorin IV (Leucovorin), Xalkori (Crizotinib), Xeloda (Capecitabine), Xospata (Gilteritinib), Xtandi (Enzalutamide), Yervoy (Ipilimumab), Yescarta (Axicabtagene), Yondelis (Trabectedin), Zaltrap (Ziv-aflibercept), Zanosar (Streptozocin), Zejula (Niraparib), Zelboraf (Vemurafenib), Zevalin (Ibritumomab Tiuxetan), Zoladex (Goserelin), Zolinza (Vorinostat), Zometa (Zoledronic acid) Zortress (Everolimus), Zydelig (Idelalisib), Zykadia (Ceritinib), Zytiga (Abiraterone), or combinations thereof. In some embodiments, the chemotherapeutic drug is selected from: Abraxane (Paclitaxel Albumin-stabilized Nanoparticle Formulation), Afinitor (Everolimus), Erlotinib Hydrochloride, Everolimus, Gemcitabine Hydrochloride, Irinotecan Hydrochloride, Lynparza (Olaparib), Mitomycin, Olaparib, cyclophosphamide, doxorubicin, oxaliplatin, mitoxantrone, Sunitinib Malate, or combinations thereof. In some embodiments, the chemotherapeutic drug is a combination of any of the aforementioned chemotherapeutic drugs.

### Certain Uses

In certain embodiments, the present invention provides a use of the composition as described herein for the manufacture of a medicament for inducing a tumor necrosis response in a subject.

In certain embodiments, the present invention provides a use of the composition as described herein for inducing a tumor necrosis response.

In certain embodiments, the present invention provides a use of the composition as described herein for the manufacture of a medicament for treating a disease or disorder in a subject.

### Compositions

Certain embodiments of the invention provide for a composition as described herein for use in medical therapy.

Certain embodiments of the invention provide the use of a composition as described herein for the manufacture of a medicament for inducing an immune response in a subject. In some embodiments, the subject is a mammal, which can include or exclude a human.

Certain embodiments of the invention provide the use of a composition as described herein for the manufacture of a medicament for inducing an immune response in a subject in combination with at least one therapeutic agent. In some embodiments, the subject is a mammal, which can include or exclude a human.

Certain embodiments of the invention provide a composition as described herein for inducing an immune response.

Certain embodiments of the invention provide a composition as described herein for inducing an immune response, in combination with at least one therapeutic agent.

Certain embodiments of the invention provide the use of a composition as described herein for the manufacture of a medicament for treating a disease or disorder in a subject.

Certain embodiments of the invention provide the use of a composition as described herein for the manufacture of a medicament for treating a disease or disorder in a subject, in combination with at least one therapeutic agent.

Certain embodiments of the invention provide a composition as described herein for the prophylactic or therapeutic treatment a disease or disorder.

Certain embodiments of the invention provide a composition as described herein for the prophylactic or therapeutic treatment of a disease or disorder, in combination with at least one therapeutic agent.

In some embodiments, the disease or disorder is a condition that requires a boost of the host immunity. In some embodiments, the disease or disorder is cancer. In some embodiments, the disease or disorder is an infectious disease.

In some embodiments, the cancer is carcinoma, lymphoma, blastoma, sarcoma, or leukemia. In some embodiments, the cancer is a solid tumor cancer.

In some embodiments, the cancer is squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer (which can include or exclude endocrine resistant breast cancer), colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia, or head and neck cancer. In some embodiments, the cancer is breast cancer.

In some embodiments, the cell is a myeloid-derived suppressor cell (MDSC) or tumor-associated macrophages M2 (TAM2).

The combinations of the present disclosure can be used to regulate tumor microenvironment, and in cancer immunotherapy. Examples of the cancer includes, but are not limited to, glioblastoma, liver cancer (such as hepatocellular carcinoma), colorectal carcinoma, glioblastoma, gastric cancer, colorectal cancer, esophageal cancer, lung cancer (such as non-small cell lung cancer (NSCLC) and small cell lung cancer), pancreatic cancer, renal cell carcinoma, benign prostate hyperplasia, prostate cancer, ovarian cancer, melanoma, breast cancer, chronic lymphocytic leukemia (CLL), Merkel cell carcinoma, Non-Hodgkin lymphoma, acute myeloid leukemia (AML), gallbladder cancer, cholangiocarcinoma, urinary bladder cancer, and uterine cancer.

### 5-Fluorouracil (5FU) Analogues

The 5FU analogues of this disclosure include those which share the core chemical structure of 5-fluorouracil such that they can be covalently connected to a RNA nucleic acid sequence in the RNAOG. In some embodiments, the 5FU analogues can include or exclude: Capecitabine, UFT, tegafur, carmofur, U-332 or floxuridine.

Capecitabine is a fluoropyrimidine carbamate which is a prodrug of 5′-deoxy-5-fluorouridine (5-DFUR), and is converted to 5-FU under physiological conditions. Capecitabine has better bioavilability than 5-FU and is readily absorbed from the gastrointestinal tract. Liver carboxylesterase hydrolyzes much of Capecitabine to 5′-deoxy-5-fluorocytadine (5′-DFCR). Cytidine deaminase, an enzyme present in most tissues, including tumors, converts 5′-DFCR to 5-DFUR. Another enzyme, thymidine phosphorylase, also present in most tissues and expressed in high amounts in many carcinomas, hydrolyzes 5′-DFUR to 5-FU.

UFT consists of 1-(2-tetrahydrofuryl)-5-fluorouracil (also referred to as tegafur, ftorafur, or FT) and uracil in a 1:4 molar concentration. UFT is also a prodrug of 5-FU. Like capecitabine, tegafur has high bioavailability and is essentially completely absorbed after oral administration and undergoes gradual hepatic conversion to 5-FU. When coadministered with uracil, UFT inhibits the degradation of 5-FU to a-fluoro-B-alanine which preferentially increases the concentration of 5-FU in tumor cells compared to that in plasma or normal tissues.

Eniluracil is a combination of 5-FU and an irreversible dihydropyrimidine dehydrogenase (DPD) inactivator. DPD is the first enzyme in the degradative pathway of pyrimidine bases. Therefore, by inhibiting the degradation of 5-FU, eniluracil increases the half-life of 5-FU, simulating the effect of a continuous infusion.

### Linkages

The linkage between the agent(s) and the RNA origami (RNAOG) nanostructure (or “nanostructure”) connects the RNAOG and the agent and does not interfere with the function of the agent or the RNAOG. In some embodiments, chemistries that link the agent to an oligonucleotide can include or exclude disulfide linkages, amino linkages, and covalent linkages. In some embodiments, the linker can include or exclude aliphatic or ethylene glycol linkers. In some embodiments the linker can include or exclude phosphodiester, phosphorothioate and/or other modified linkages. In some embodiments, the linker is a binding pair. As used herein, the term “binding pair” refers to two molecules which interact with each other through any of a variety of molecular forces which can include or exclude ionic, covalent, hydrophobic, van der Waals, and hydrogen bonding, so that the pair have the property of binding specifically to each other. Specific binding means that the binding pair members exhibit binding to each other under conditions where they do not bind to another molecule. Binding pairs can include or exclude biotin-avidin, hormone-receptor, receptor-ligand, enzyme-substrate probe, IgG-protein A, antigen-antibody, aptamer-target and the like. In some embodiments, a first member of the binding pair comprises avidin or streptavidin and a second member of the binding pair comprises biotin. In some embodiments, a first member of the binding pair comprises nickel and a second member of the binding pair comprises a His-tag. In some embodiments, the binding pair is another affinity ligand interaction.

### Formulations

The pharmaceutical combination of the present invention may be formulated with a “carrier.” As used herein, “carrier” includes any solvent, dispersion medium, vehicle, coating, diluent, antibacterial and/or antifungal agent, isotonic agent, absorption delaying agent, buffer, carrier solution, suspension, colloid, and the like. The use of such media and/or agents for pharmaceutical active substances is well known in the art. For example, the pharmaceutical combinations can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, lotion, gel, ointment, or a controlled-release patch or spray applied to the skin; (4) intravaginally or intrarectally, for example, as a pessary, cream, suppository or foam; (5) sublingually; (6) ocularly; (7) transdermally; (8) transmucosally; or (9) nasally.

The pharmaceutical combination of the present disclosure may be provided in a single formulation. In other embodiments, the pharmaceutical combination of the present disclosure may be provided in separate formulations. A pharmaceutical combination may be formulated in a variety of and/or a plurality of forms adapted to one or more preferred routes of administration. Thus, a pharmaceutical combination can be administered via one or more known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical combination, or a portion thereof, can be administered to a mucosal surface, such as by administration to, for example, the nasal or respiratory mucosa (e.g., by spray or aerosol). A pharmaceutical combination, or a portion thereof, also can be administered via a sustained or delayed release.

A pharmaceutical combination of the present disclosure may be conveniently presented in unit dosage form and may be prepared by methods well known in the art of pharmacy. Methods of preparing a combination with a pharmaceutically acceptable carrier include the step of bringing the pharmaceutical combination of the present disclosure into association with a carrier that constitutes one or more accessory ingredients. In general, a pharmaceutical combination of the present disclosure may be prepared by uniformly and/or intimately bringing the active compound into association with a liquid carrier, a finely divided solid carrier, or both, and then, if necessary, shaping the product into the desired formulations.

In some embodiments, the method can include administering a sufficient amount of the pharmaceutical combination of the present disclosure to provide a dose of, for example, from about 0.1 mg/kg to about 1,000 mg/kg to the subject. In some embodiments, the dosage amount of the RNAOG and 5FU or 5FU analogue can be from about 0.1 mg/kg to about 1 mg/kg, from about 1 mg/kg to about 10 mg/kg, from about 10 mg/kg to about 100 mg/kg, or from about 100 mg/kg to about 1,000 mg/kg to the subject per day.

### Administration

As described herein, methods of the invention comprise administering a composition comprising a composition as described herein. In some embodiments, such compositions are formulated as a pharmaceutical composition and administered to a mammalian host, which can include or exclude a human patient in a variety of forms adapted to the chosen route of administration, i.e., orally or parenterally, by intravenous, intramuscular, intraperitoneal or topical or subcutaneous routes.

In some embodiments, he compositions are systemically administered, e.g., orally, in combination with a pharmaceutically acceptable vehicle which can include or exclude an inert diluent or an assimilable edible carrier. The compositions may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. In some embodiments, such compositions and preparations comprise at least 0.1% of active RNAOG with 5FU or 5FU analogue. The percentage of the compositions and preparations may, of course, be varied and may conveniently be between about 0.1 to about 60% of the weight of a given unit dosage form. The amount of active compound in such therapeutically useful compositions is such that an effective dosage level will be obtained. The tablets, troches, pills, capsules, and the like may also contain the following: binders which can include or exclude gum tragacanth, acacia, corn starch or gelatin; excipients which can include or exclude dicalcium phosphate; a disintegrating agent which can include or exclude corn starch, potato starch, alginic acid and the like; a lubricant which can include or exclude magnesium stearate; and a sweetening agent which can include or exclude sucrose, fructose, lactose or aspartame or a flavoring agent which can include or exclude peppermint, oil of wintergreen, or cherry flavoring may be added. When the unit dosage form is a capsule, it may contain, in addition to materials of the above type, a liquid carrier, which can include or exclude a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring which can include or exclude cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. In some embodiments, the liquid carrier or vehicle can be a solvent or liquid dispersion medium comprising a liquid which can include or exclude: water, ethanol, a polyol (for example, glycerol, propylene glycol, liquid polyethylene glycols, and the like), vegetable oils, nontoxic glyceryl esters, and suitable mixtures thereof. The proper fluidity can be maintained by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Thickeners which can include or exclude synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver a compound to the skin can include or exclude: Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), each of which are herein incorporated by reference in their entirety.

Useful dosages of compounds can be determined by comparing their in vitro activity, and in vivo activity in animal models. Methods for the extrapolation of effective dosages in mice, and other animals, to humans can include U.S. Pat. No. 4,938,949, herein incorporated by reference.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compound may be conveniently formulated in unit dosage form. In one embodiment, the invention provides a composition comprising a compound formulated in such a unit dosage form. The desired dose may conveniently be presented in a single dose or as divided doses administered at appropriate intervals. In some embodiments, the dose interval is selected from two, three, four or more sub-doses per day.

### EXAMPLES

The invention will now be illustrated by the following non-limiting Examples.

### Example 1

### Synthesis of a Representative RNAOG

The synthesis of a representative RNAOG having a square sheet shape is similar to that described previously (US20210246452).

### Design and Synthesis of RNAOG

To synthesize long ssRNA molecules, a DNA template with both T7 and T3 promoter sequences was first synthesized as two fragments. The two DNA fragments were subcloned into a vector through Eco RI and Hind III restriction sites and amplified in E. coli. The purified plasmids were then linearized by Eco RI and Hind III, and transcribed using T7 RNA polymerase and/or T3 RNA polymerase. The in vitro transcribed RNA molecules were then purified, self-folded from 65° C. to 25° C. with a 1° C. per 15 minutes ramp. The RNA molecules were, characterized with AFM.

In one embodiment, a design of an 8 nt helical domain, followed by an 8 nt locking domain, followed by a 9 nt helical domain, followed by an 8 nt locking domain (i.e., an 8-8-9-8 structure) was designed, which gives three turns per 33-bp repeating unit. Using the 8-8-9-8 design, an 1868-nt rectangle RNAOG was constructed. The RNA strand for 1868-nt rectangle from both the sense strand and the antisense strand were tested and both produced expected and identical shapes under AFM.

Representative RNAOG structures were constructed from ssRNA with synthetic sequence ranging in length from ˜1000 to ˜10,000 nt. The RNAOG uses no auxiliary strands and can be designed to form a wide variety of space-filling compact shapes. The represenative RNAOG described herein is a purely de novo designed structure that does not rely on the availability of highly sequence specific, naturally occurring molecular interaction motifs with defined geometrical arrangements (for example, the RNA kissing loops) and thus promises, in principle, better designability and scalability, as reflected in practice by construction of a 6000-nt ssRNA structure.

### Materials and Methods

Within this application, unless otherwise stated, the techniques utilized may be found in any of several well-known references such as: Molecular Cloning: A Laboratory Manual (Sambrook, et al., 1989, Cold Spring Harbor Laboratory Press), Current Protocols in Molecular Biology (Ausebel et al., Wiley-Interscience, 1988. New York), and PCR Protocols: A Guide to Methods and Applications (Innis, et al. 1990. Academic Press, San Diego, Calif.).

In certain embodiments, the methods of this disclosure can treat cancer in a relevant model. Mouse models for pancreatic cancer to which the methods of this disclosure are expected to demonstrate the treatment of cancer are described in Herreros-Villanueva et al., Mouse models of pancreatic cancer World J Gastroenterol. (2012) March 28; 18 (12): 1286-1294. doi: 10.3748/wjg.v18.i12.1286, PubMed ID: 22493542), incorporated herein by reference.

Restriction endonucleases EcoRI (5,000 units), Xhol (5,000 units) and HindIII (5,000units), T7 and T3 RNA polymerases (5,000 units), NEB 10-beta competent E. coli were purchased from NEW ENGLAND BIO LABS INC. Pureyield plasmid miniprep system and the Wizard SV Gel and PCR Clean-UP System were purchased from Promega (www.promegA.com). RNA Clean and Concentrator-25 was purchased from Zymo Research (www.zymoresearch.com).

### DNA and RNA Sequence Design

Representative DNA sequences were designed with the Tiamat software (S. Williams, K. Lund, C. Lin, P. Wonka, S. Lindsay, H. Yan, Tiamat: A three-dimensional editing tool for complex DNA structures, in International Workshop on DNA-Based Computers (Springer, 2008), pp. 90-101). Sequence generation of RNA ssOrigami structures uses the following criteria in the software: (1) Unique sequence limit: 8-10; (2) Repetition limit: 8; (3) G repletion limit: 4; (4) G/C percentage: 0.38-0.5. For RNAOG sequences, T7/T3 promoter sequences followed with two or three consecutive Gs were added to the end to facilitate efficient in vitro transcription reactions.

### In Vivo Cloning Sample Preparation

The DNA templates for transcribing ssRNAs were divided into two DNA sequences with both T7 and T3 promoter sequences added to the ends, and ordered as gene synthesis products from BioBasic Inc. The two fragments were then subcloned into pUC19 vector using the same restriction sites as ssDNA origami. The final plasmids were linearized by EcoRI and HindIII, and transcribed by T7 or T3 RNA polymerase following manufacturer's instruction (New England Biolabs). The transcription reaction mixture was purified by RNA Clean and Concentrator kit as described in the manufacturer's instruction (Zymo Research). After purification, the ssRNA was annealed using the same program as ssDNA origami.

### AFM Imaging

For AFM imaging, the sample (15 mL) was deposited onto a freshly cleaved mica surface (Ted Pella, Inc.) and left to adsorb for 1 minute. 40 mL 1×TAE-Mg2+ and 2-15 mL 100 mM NiCl2 was added onto the mica, and the sample was scanned on a Veeco 5 Multimode AFM in the Scanasyst in Fluid mode using scanasyst in fluid+tips (Vecco, Inc.).

Synthesis and Replication of ssRNA for RNAOG Folding.

The DNA templates for transcribing ssRNAs were divided into two DNA sequences with both T7 and T3 promoter sequences added to the ends, and ordered as gene synthesis products from BioBasic Inc. The two fragments were then subcloned into pUC19 vector using the same restriction sites as ssDNA origami. The final plasmids were linearized by EcoRI and HindIII, and transcribed by T7 or T3 RNA polymerase following manufacturer's instruction (New England Biolabs). The transcription reaction mixture was purified by RNA Clean & Concentrator kit as described in the manufacturer's instruction (Zymo Research). After purification, the ssRNA was annealed using the same program as ssDNA origami, and characterized by AFM.

### DNA Template Used to Generate the RNAOG Nanostructures

Forward strand: SEQ ID NO: 14. Reverse strand: SEQ ID NO: 15

### Example 2

5FU and RNAOG combined immunochemotherapy for pancreatic cancer treatment

Pancreatic ductal adenocarcinoma (PDA) is one of the most immune-resistant tumor types. Single-agent immune modulators targeting immune checkpoint blockade and multi-modal therapies including target immunotherapy have been proven to be clinically ineffective. Intensive studies to explore novel targeted and combination therapeutic strategies are urgently needed. Recently, nucleic acid nanotechnology has emerged as a promising approach for cancer targeting and treatment. Replicable single-strand RNA origami (RNA-OG) technology stimulates a potent innate response primarily through a Toll-like receptor (TLR3) pathway to activate NK-and CD8-dependent antitumor immunity and counteract the peritoneal immunosuppressive environment in a murine peritoneal colon cancer model. The pancreatic tumor microenvironment includes robust innate immune suppressor cell types such as myeloid-derived suppressor cells (MDSC) and tumor-associated macrophages M2 (TAM2) in the tumor microenvironment. This example describes a combination therapy using the RNAOG with a representative chemotherapy drug such as 5fluorouricil (5FU), as two immunochemotherapeutic strategies to efficiently target tumor-associated macrophage (TAM) and kill pancreatic cancer cells in tumor microenvironments.

### 5FU-RNAOG Synthesis and Characterization

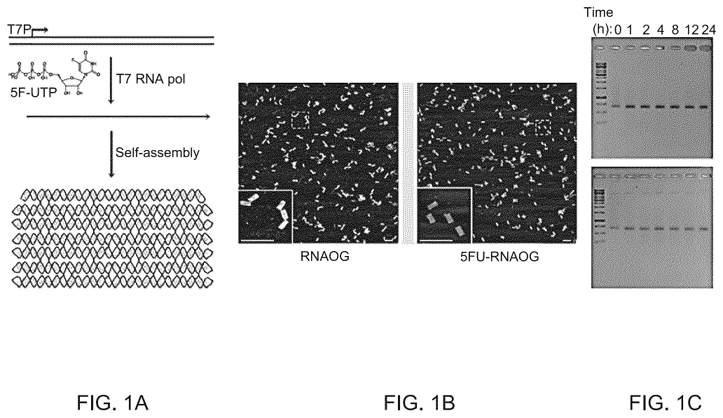

The 5FU-RNAOG was synthesized in a similar approach for the RNAOG synthesis as described in Example 1. UTP was substituted by 5F-UTP during the RNA in vitro transcription reaction so that the 5FU was covalently linked to the RNAOG nanostructures (FIG. 1A). The 5FU-RNAOG, upon internalization into the tumor cells, will be degraded, thereby releasing the 5FU which will inhibit the tumor cell growth. Both RNAOG and 5FU-RNAOG assembled into the same rectangular shaped structure as shown in the AFM images (FIG. 1B). The serum stability was performed by incubating RNAOG (FIG. 1C top) and 5FU-RNAOG (FIG. 1C bottom) in 10% FBS at 37° C. Both structures remained to be intact even after 24 hours incubation as indicated by gel electrophoresis.

### In Vitro Pancreatic Cancer Cell Growth Inhibition Assay

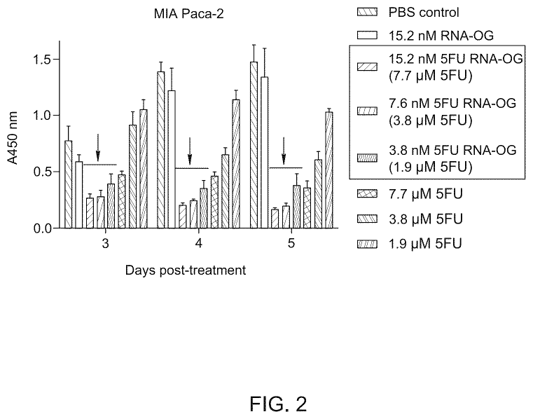

To evaluate the tumor cell inhibition with the 5FU and 5FU-RNAOG, a pancreatic cancer cell growth inhibition assay was performed. The pancreatic cancer cell line, MiaPaca-2, was cultured in 96-well plates, and supplemented with different doses of 5FU or 5FU-RNAOG, as indicated in FIG. 2. The cell counting Kit-8 was utilized to quantitate the viable cell numbers. As shown in FIG. 2, the 5FU-RNAOG significantly inhibits the pancreatic cancer cell proliferation comparing with the 5FU at the equivalent dosages.

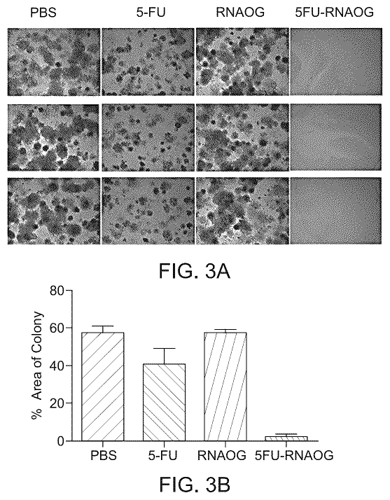

To further demonstrate the tumor cell regression effect of 5FU-RNAOG, a colony formation assay was performed (FIG. 3). MiaPaca-2 cells were seeded in 6-well plates with the same density. 5FU, RNAOG, and 5FU-RNAOG were introduced into the MiaPaca-2 cells which were cultured to form colonies in 1-3 weeks. Colonies were fixed with glutaraldehyde (6%, v/v), and stained with crystal violet (0.5%, w/v) and counted using a stereomicroscope. The 5FU-RNAOG significantly inhibited the MiaPaca-2 cell colony formation comparing with other treatments (FIG. 3).

In vivo efficacy of 5FU-RNAOG on Miapaca pancreatic cancer cell line

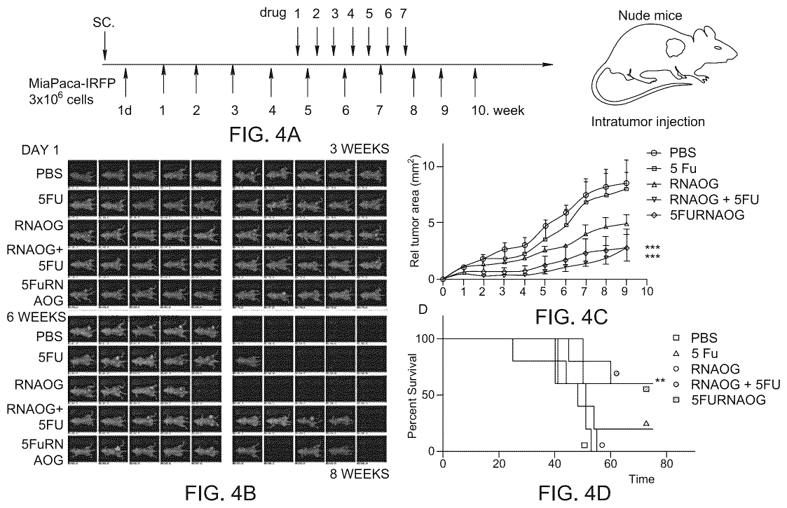

The in vitro tumor cell suppression effect of 5FU-RNAOG justified further investigation as to whether the combination of the RNAOG with the anticancer drug 5-fluorouracil (5FU) exerts anti-tumor activity in vivo. The human pancreatic cancer cell line, MiaPaca-2, was used as the model because it forms solid tumors in nude mice upon subcutaneous injection (SC). Specifically, MiaPaca-2 cells were engineered to express near infrared fluorescent protein (iRFP) which allows for real-time monitoring of tumor growth of live animals, especially when the tumor load is low. Four weeks following SC administration of MiaPaca-2-iRFP cells, the mice received the first intratumor treatment on day 0, and were treated twice a week for a total 7 times (FIG. 4A). Tumor progression was monitored via the fluorescent intensity of iRFP (FIG. 4B), as well as the directly measured tumor size (FIG. 4C). The PBS and 5FU treated mice had developed large tumors. In contrast, both 5FU-RNAOG and RNAOG+5FU (physical mixture of RNAOG and 5FU) exhibit significant tumor growth regression. Furthermore, the mice survival data showed that both 5FU-RNAOG and RNAOG+5FU treatments offered superior increased survival over the PBS and 5FU groups (FIG. 4D). This finding indicates that the combination therapy of RNAOG and 5FU induces an effective tumor-inhibitory effect against pancreatic cancer. Both covalent conjugation (5FU-RNAOG) and physical mixture (RNAOG+5FU) showed similar and promising results. FIG. 10 shows the results of a replicate experiment, demonstrating the repeatability of the results. MiaPaca-2 animal models were obtained from Altogen Labs (Austin, TX).

rginase I expression is decreased in xenograft tumor tissue by immunohistochemical analysis

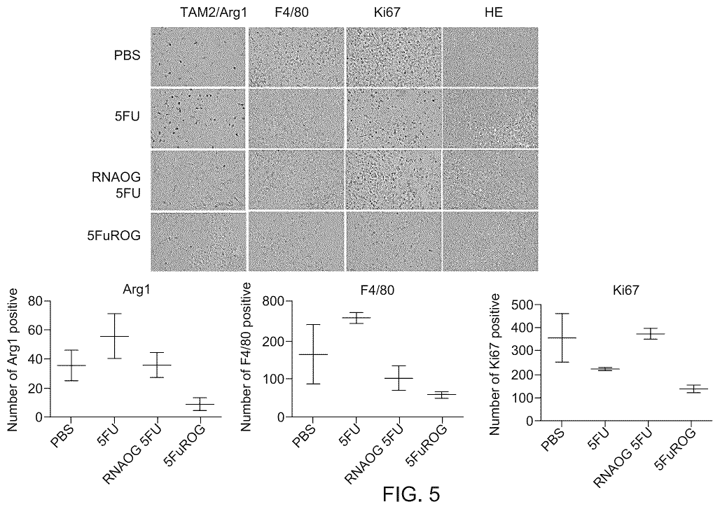

To assess the levels of tumor associate macrophage and pancreatic cancer cell growth in xenograft tumor, F4/80, Arg1, and Ki67 expression in xenograft tumor tissue was analyzed using immunohistochemistry staining. F4/80 is a glycoprotein expressed by murine macrophage as a macrophage marker. The Arg1 gene provides instruction for producing the enzyme arginase which participates in the urea cycle. In growing tumor, tumor-associated macrophages are often referred to M2-like macrophages, which are cells that display immunosuppressive and tumorigenic functions and express the enzyme arginase 1 (Arg1). The expression of Ki67 is a proliferation marker of tumor cell, which is strongly associated with tumor cell proliferation and growth. As shown in FIG. 5, both F4/80 and Ki67 markers exhibit significant high expression levels in xenograft tumor tissues in the PBS control group, indicated aggressive tumor growth. In contrast, both 5FU-RNAOG and RNAOG+5FU (physical mixture of RNAOG and 5FU) display lower expression of F4/80 marker, and 5FU-RNAOG treated tissues trigger lower expression of Arg1, which demonstrated that RNAOG decreased tumor-associated macrophage and promoted tumor growth regression. 5FU-RNAOG decrease M2-like tumor associated macrophage. Both covalent conjugation (5FU-RNAOG) and physical mixture (RNAOG+5FU) showed promising results.

### Combination of 5FU and RNAOG Inhibits Cell Growth

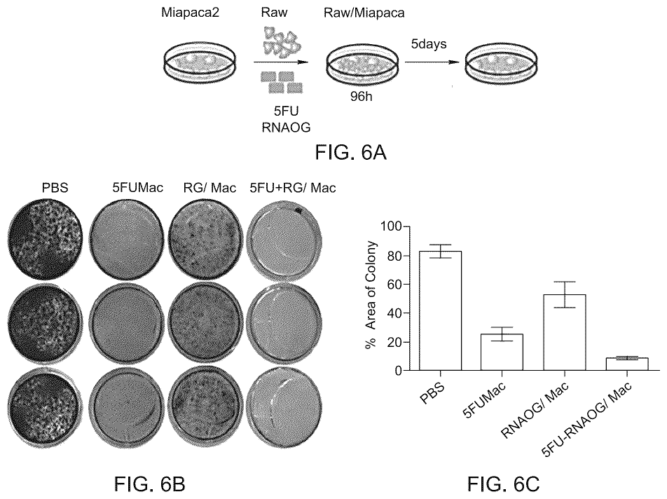

A colony formation assay was performed to investigate the cell regression effect with the combination therapy of 5FU and RNAOG (FIG. 6). MiaPaca-2 cells were seeded in 6-well plates with the same density. The PBS, 5FU+RAW264.7 (5FU/Mac), RNAOG+RAW264.7 (ROG/Mac), or 5FU+RNAOG+RAW264.7 (5FU-ROG/Mac) were introduced into the MiaPaca-2 cells. The colony assay indicated that the RNAOG significantly inhibited the pancreatic cell growth with the addition of macrophage compared with the PBS control group, while the combination of 5FU and RNAOG further reduced the cell viability (FIG. 6).

Gene Expression Level in Macrophage Cells after RNAOG Treatment

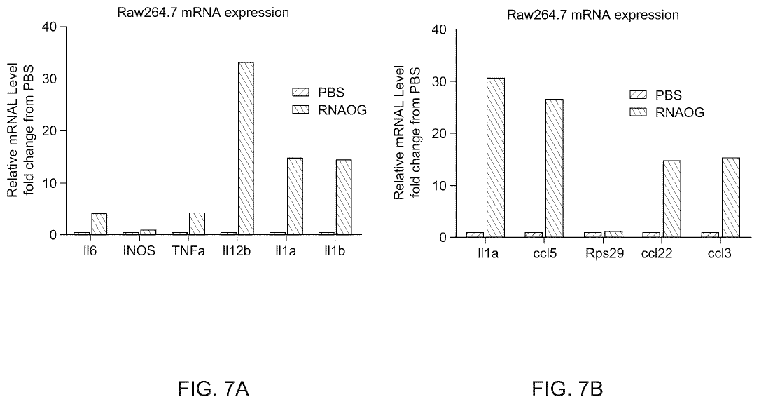

Selected gene expression levels changes in Macrophage cells with the RNAOG treatment were analyzed. M1-like TAM associated genes such as cytokine and chemokine genes were selected for measurement. The cytokine associated genes include IL6, TNFa, IL12b, IL1a and II 1b; the chemokine genes contain CCL5, CCL22 and CCl3. Real time qRT-PCR was performed to quantitate the mRNA expression levels (FIG. 7). Most of the cytokine genes were upregulated in RAW264.7 cells with the incubation of RNAOG (FIG. 7A), indicating the immune response is activated. Furthermore, the M1 type associated genes were also significantly upregulated with the RNAOG treatment (FIG. 7B).

### Internalization of RNAOG Induces Macrophage Death

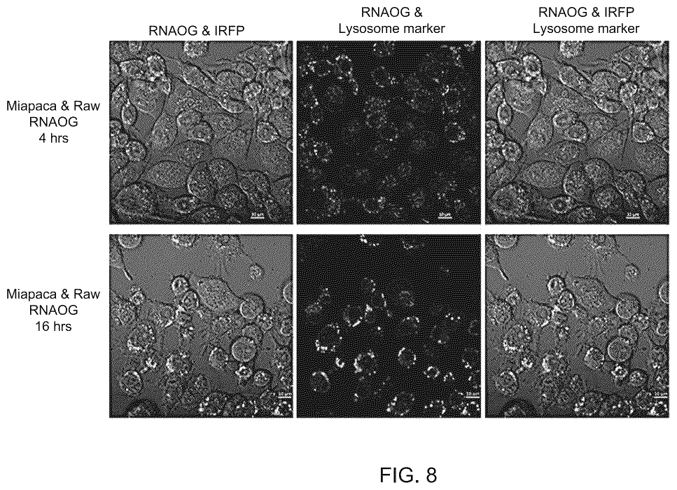

To evaluate how RNAOG works in the tumor microenvironment, the RAW264.7 cells and MiaPaca2-iRFP cells were co-cultured with the addition of AF488 labeled RNAOG. The AF488 is a representative diagnostic agent which can be connected to the RNAOG by covalent attachment by the addition of AlexaFluor488-UTP during RNA synthesis (Jena Bioscience PN RNT-101-AF488, www.jenabioscience.com). The MiaPaca2-iRFP can be distinguished with RAW264.7 with its intrinsic iRFP fluorescence. As shown in the confocal images (FIG. 8), the RNAOG was mainly internalized into RAW264.7 cells rapidly. Most of the RNAOG fluorescence colocalizes with lysotracker dye which indicates that the RNAOG internalizes into the endosomes/lysosomes of the macrophage.

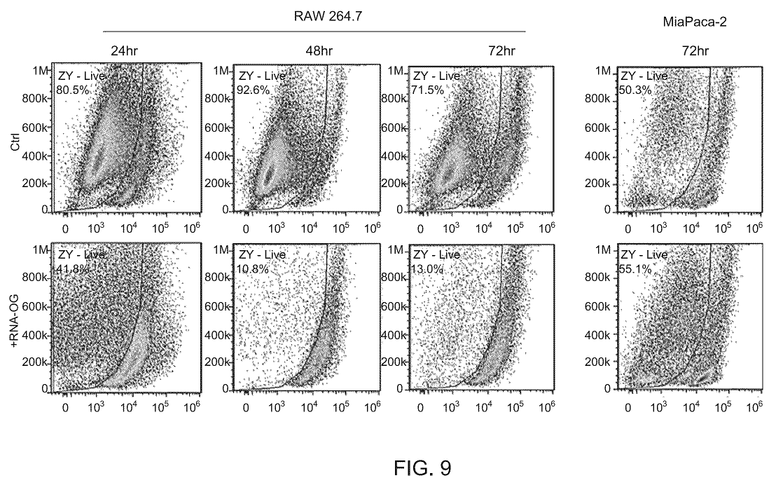

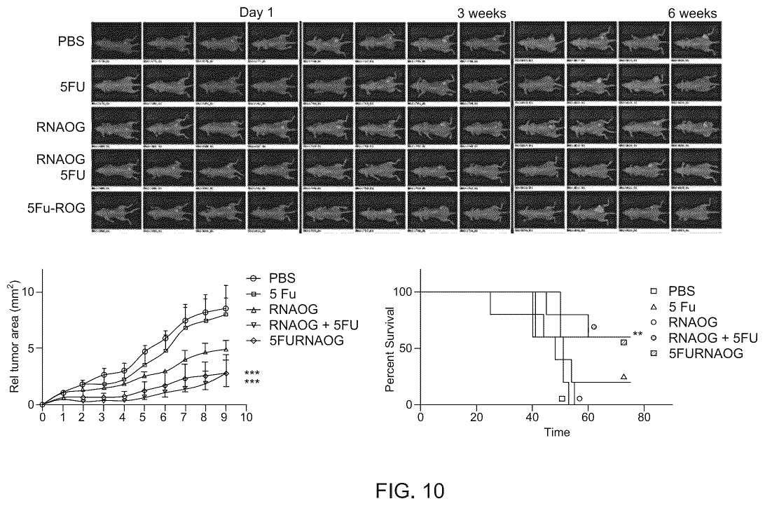

To demonstrate if RNAOG promotes the death of macrophage cells in the tumor microenvironment, the viability of RAW 264.7 cells in the co-culture with MiaPaca-2 cells was measured. The RNAOG was directly introduced into the coculture of RAW 264.7 and MiaPaca-2-iRFP cells and incubated for 24, 48, and 72 hours. The cells were harvested, stained, and analyzed by flow cytometry. The RAW 264.7 (CD11b+iRFP−) cells were separated with MiaPaca-2-iRFP (CD116−iRFP+) cells by fluorescence through the flow cytometry. Furthermore, the cell viability was analyzed with the zombie yellow dye staining in which only dead cells exhibit strong fluorescence (FIG. 9). After 48 hours of RNAOG, majority of the RAW 264.7 cells died, with only 10.8% and 13% remained alive at 48 and 72 hours, respectively. In contrast, most of the RAW 264.7 cells survived in the control group, even after 72 hours. This data indicates that the RNAOG induces the death of macrophage only in the coculture with the pancreatic tumor cells. Meanwhile, no significant viability differences were observed with the cocultured MiaPaca-2 cells in the absence or presence of RNAOG (FIG. 9). The results described herein demonstrate the RNAOG induced death of TAM M2 and MDSC cells in a tumor microenvironment. The immunochemotherapy with the combination of RNAOG with 5FU, either by covalently conjugation or physical mixing, synergistically increases the antitumor efficacy.

The examples provided herein clearly demonstrate that the compositions of this disclosure can be used to significantly reduce the tumor burden in a subject, resulting in an increased survival time, including up to the end of the experimental study.

Although the foregoing specification and examples fully disclose and enable the embodiments of the present disclosure, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of inducing an immune response a subject, comprising administering to the subject an effective amount of a RNAOG and 5FU or a 5FU analogue.

2. A method of treating a disease or disorder in a subject, comprising administering to the subject in need thereof a therapeutically effective amount of a RNAOG and 5FU or a 5FU analogue.

3. The method of claim 2, wherein the disease or disorder is cancer.

4. The method of claim 3, wherein the cancer is pancreatic cancer.

5. The method of claim 2, wherein the 5FU analogue is selected from capetabine, UFT, tegafur, carmofur, U-332 or floxuridine.

6. The method of claim 4, further comprising administering at least one therapeutic agent to the subject.

7. The method of claim 6, wherein the therapeutic agent is a chemotherapeutic drug.

8. The method of claim 7, wherein the chemotherapeutic drug is selected from: Abraxane (Paclitaxel Albumin-stabilized Nanoparticle Formulation), Afinitor (Everolimus), Erlotinib Hydrochloride, Everolimus, Gemcitabine Hydrochloride, Irinotecan Hydrochloride, Lynparza (Olaparib), Mitomycin, Olaparib, cyclophosphamide, doxorubicin, oxaliplatin, mitoxantrone, or Sunitinib Malate.

9. The method of claim 2, wherein the 5FU or 5FU analogue is covalently connected to the RNAOG sequence.

10. The method of claim 2, wherein the 5FU or 5FU analogue is not covalently connected to the RNAOG sequence.

11. A method of reducing the proliferation of a cancer tumor cell, the method comprising contacting said cancer tumor cell with a RNAOG and 5FU or a 5FU analogue.

12. The method of claim 11, wherein the 5FU or 5FU analogue is covalently connected to the RNAOG sequence.

13. The method of claim 11, wherein the 5FU analogue is selected from capetabine, UFT. tegafur, carmofur, U-332 or floxuridine.

14. The use of a RNAOG and 5FU or a 5FU analogue for the manufacture of a medicament for inducing an immune response in a subject.

15. A composition comprising RNAOG, 5FU, and a pharmaceutically acceptable carrier.

16. The composition of claim 15, wherein the RNAOG is covalently linked to 5FU.

17. The use of a composition comprising RNAOG covalently linked to 5FU for the manufacture of a medicament for treating a disease or disorder in a subject.

18. A composition comprising RNAOG covalently linked to 5FU for the prophylactic or therapeutic treatment of a disease or disorder in a subject.

19. A kit comprising a composition comprising RNAOG covalently linked to 5FU and instructions for administering the composition to a subject to induce an immune response or to treat a disease or disorder.

20. The kit of claim 15, further comprising at least one therapeutic agent.
